(19) United States
(12) Patent Application Publication
(10) Pub. No.: US 2024/0141355 A1
(43) Pub. Date: May 2, 2024

(54) MODULATION OF GYS1 EXPRESSION

(71) Applicants: Ionis Pharmaceuticals, Inc., Carlsbad, CA (US); The Hospital for Sick Children, Toronto (CA)

(72) Inventors: Tamar R. Grossman, La Jolla, CA (US); Susan M. Freier, San Diego, CA (US); Berge Minassian, Toronto (CA); Saija Ahonen, Toronto (CA)

(21) Appl. No.: 18/331,311

(22) Filed: Jun. 8, 2023

Related U.S. Application Data
(63) Continuation of application No. 17/561,095, filed on Dec. 23, 2021, now Pat. No. 11,713,462.
Application No. 17/561,095 is a division of application No. 16/306,831, filed on Dec. 3, 2018, now Pat. No. 11,236,339.
(60) Provisional application No. 62/430,106, filed on Dec. 5, 2016.
(60) Provisional application No. 62/351,396, filed on Jun. 17, 2016.

(51) Int. Cl. C12N 15/113; A61K 31/7088; A61K 45/06; A61P 3/08; A61P 25/28

(52) U.S. Cl. CPC C12N 15/1137; A61K 31/7088; A61K 45/06; A61P 3/08; A61P 25/28; C12N 15/113; C12N 2310/11; C12N 2310/315; C12N 2310/321; C12N 2310/3341; C12N 2310/341; C12N 2320/30

(57) ABSTRACT

Provided herein are methods, compounds, and compositions for reducing expression of GYS1 in an individual. Such methods, compounds, and compositions are useful to treat, prevent, delay, or ameliorate a glycogen storage disease or disorder in an individual in need.

### SEQUENCE LISTING

The present application is being filed along with a Sequence Listing in electronic format. The Sequence Listing is provided as a file entitled BIOL0294WOSEQ_ST25.txt, created on Jun. 12, 2017 which is 108 KB in size. The information in the electronic format of the sequence listing is incorporated herein by reference in its entirety.

### FIELD OF THE INVENTION

Provided herein are methods, compounds, and compositions useful for reducing expression of glycogen synthase 1 (hereinafter referred to as GYS1) in an animal. Also, provided herein are methods, compounds, and compositions comprising GYS1 inhibitors, which can be useful in reducing GYS1-related diseases or conditions in an animal. Such methods, compounds, and compositions can be useful, for example, to treat, prevent, delay or ameliorate a glycogen storage disease or a polyglucosan disorder in an animal.

### BACKGROUND

Glycogen is a branched polymer of glucose that constitutes the sole carbohydrate reserve for mammals. It is synthesized by glycogen synthase (GYS), the only mammalian enzyme able to polymerize glucose (Bollen M. et al. Biochem. J. 1998 336: 19-31). Glycogen biosynthesis involves chain elongation by glycogen synthase and chain branching by glycogen branching enzyme. If chain elongation outbalances chain branching, glycogen forms starch-like precipitates made up of long, non-branched chains called polyglucosans. The most glycogenic tissues are muscle and liver.

Glycogen synthase 1 (GYS1) is an enzyme involved in converting glucose to glycogen by catalyzing the elongation of short glucose polymers into long glycogen polymers. Mutations in GYS1 are associated with glycogen storage diseases. In the brain, glycogen is normally stored in astrocytes (brown A. M. J. Neurochem. 89: 537-552, 2004) and glycogen synthesis is normally absent in neurons because of tight regulation of GYS1 by laforin and malin (Vilchez et al., Nat. Neurosci. 10: 1407-1413, 2007). Nevertheless, aberrant glycogen accumulation in neurons is a hallmark of patients suffering from Lafora disease, Pompe disease, Andersen's disease, adult polyglucosan disease, or other polyglucosan disorders.

Currently, there is a lack of acceptable options for treating glycogen storage diseases. There is also a lack of specific inhibitors for glycogen synthase. It is therefore an object herein to provide methods for the treatment of such diseases.

### SUMMARY

Provided herein are compositions, compounds and methods for modulating expression of GYS1-associated diseases such as glycogen storage disease and polyglucosan disorders, including Lafora disease, adult polyglucosan body disease, Andersen's disease, and Pompe disease. In certain embodiments, these compositions, compounds and methods are for modulating the expression of GYS1. In certain embodiments, the GYS1 modulator is a GYS1-specific inhibitor. In certain embodiments, the GYS1-specific inhibitor decreases expression or activity of GYS1. In certain embodiments, GYS1-specific inhibitors include nucleic acids, proteins and small molecules. In certain embodiments, the GYS1-specific inhibitor is a nucleic acid. In certain embodiments, GYS1-specific inhibitor comprises a modified oligonucleotide. In certain embodiments, the modified oligonucleotide can be single stranded or double stranded.

Certain embodiments are directed to novel GYS1 inhibitors useful for inhibiting GYS1, which can be useful for preventing, inhibiting, or slowing the progression of accumulation of glycogen in tissues. Certain embodiments are directed to GYS1 inhibitors useful for treating, ameliorating, or slowing progression of polyglucosan disorders, including, but not limited to, Lafora disease, adult polyglucosan body disease, and Pompe disease. Certain embodiments relate to the novel findings of antisense inhibition of GYS1 resulting in reduction of glycogen synthesis, glycogen aggregation, reduction of Lafora bodies accumulation, reduction of polyglucosan bodies, glycogen level normalization, reduction or cessation of seizures, improvement or prevention of cognitive deterioration, reduction of neuromuscular weakness, and reduction or cessation of epileptic episodes. Certain embodiments are directed to GYS1 inhibitors useful in improving glycogen levels. Certain embodiments are directed to GYS1 inhibitors useful in reducing incidence of seizures or epileptic episodes.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ISIS number (ISIS #) indicate a combination of nucleobase sequence, chemical modification, and motif.

Unless otherwise indicated, the following terms have the following meanings:
“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).
“2′-O-methoxyethyl” (also 2′-MOE and 2′-O(CH₂)₂—OCH₃) refers to an O-methoxy-ethyl modification at the 2′ position of a furanosyl ring. A 2′-O-methoxyethyl modified sugar is a modified sugar.
“2′-MOE nucleoside” (also 2′-O-methoxyethyl nucleoside) means a nucleoside comprising a 2′-MOE modified sugar moiety.
“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.
“3′ target site” refers to the nucleotide of a target nucleic acid which is complementary to the 3′-most nucleotide of a particular compound.
“5′ target site” refers to the nucleotide of a target nucleic acid which is complementary to the 5′-most nucleotide of a particular compound.
“5-methylcytosine” means a cytosine with a methyl group attached to the 5 position.
“About” means within ±10% of a value. For example, if it is stated, “the compounds affected about 70% inhibition of a GYS1”, it is implied that GYS1 levels are inhibited within a range of 60% and 80%.
“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.
“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.
“Adult polyglucosan body disease” is characterized by dysfunction of the central and peripheral nervous systems. Associated symptoms and findings may include sensory loss in the legs, progressive muscle weakness of the arms and legs, gait disturbances, urination difficulties, and/or cognitive impairment or dementia.
“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.
“Andersen's disease”, also known as glycogen storage disease type IV, is caused by deficient activity of the glycogen-branching enzyme, resulting in accumulation of abnormal glycogen in the liver, muscle, and other tissues. The disease course is typically characterized by progressive liver cirrhosis and liver failure. In some case, several neuromuscular variants of Andersen's disease occur that may be evident at birth, late childhood, or adulthood.
“Animal” refers to a human or non-human animal, including, but not limited to, mice, rats, rabbits, dogs, cats, pigs, and non-human primates, including, but not limited to, monkeys and chimpanzees.
“Antisense activity” means any detectable and/or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.
“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.
“Antisense inhibition” means reduction of target nucleic acid levels in the presence of an antisense compound complementary to a target nucleic acid compared to target nucleic acid levels in the absence of the antisense compound.
“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.
“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.
“Ataxia” means the loss of full control of bodily movements.
“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.
“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.
“Chemical modification” in a compound describes the substitutions or changes through chemical reaction, of any of the units in the compound. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.
“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.
“Chimeric antisense compounds” means antisense compounds that have at least 2 chemically distinct regions, each position having a plurality of subunits.
“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.
“Cleavable moiety” means a bond or group of atoms that is cleaved under physiological conditions, for example, inside a cell, an animal, or a human.
“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (NC) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.
“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.
“Dementia” means a continued loss of intellectual function that impairs memory, judgment, and thought.
“Designing” or “Designed to” refer to the process of designing a compound that specifically hybridizes with a selected nucleic acid molecule.
“Diluent” means an ingredient in a composition that lacks pharmacological activity, but is pharmaceutically necessary or desirable. For example, the diluent in an injected composition can be a liquid, e.g. saline solution.
“Differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.
“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.
“Dosing regimen” is a combination of doses designed to achieve one or more desired effects.
“Double-stranded compound” means a compound comprising two oligomeric compounds that are complementary to each other and form a duplex, and wherein one of the two said oligomeric compounds comprises an oligonucleotide.
“Effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.
“Efficacy” means the ability to produce a desired effect.
“Ensembl ID’ is an identification number consisting of letters and numbers assigned to a gene sequence by Ensembl, which is a joint project between EMBL-EBI and the Wellcome Trust Sanger Institute to develop a software system that produces and maintains automatic annotation of selected eukaryotic genomes. Ensembl annotation helps identify a gene location in a particular genome and can be used to configure the equivalent gene on another species' genome.
“Epilepsy” is a central nervous system disorder in which nerve cell activity in the brain becomes chronically disrupted. In certain instances, it may cause seizures, periods of unusual behavior, sensations, and sometimes loss of consciousness. In certain instances, it may also cause other symptoms including myoclonus, cognitive deficits, learning disabilities, or developmental delay in children. In certain instances, it may lead to death in some patients. In certain instances, some forms of epilepsy are associated with progressive neurodegenerative diseases. Many people with epilepsy have more than one symptom.
“Expression” includes all the functions by which a gene's coded information is converted into structures present and operating in a cell. Such structures include, but are not limited to the products of transcription and translation.
“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”
“Glycogen” is a polysaccharide that is the principal storage form of glucose in animals. Glycogen is found in the form of granules in the cystosol in a variety of tissues, including brain.
“GYS1” means glycogen synthase 1 and refers to any nucleic acid of GYS1. For example, in certain embodiments, GYS1 includes a DNA sequence encoding GYS1, an RNA sequence transcribed from DNA encoding GYS1 (including genomic DNA comprising introns and exons). The target may be referred to in either upper or lower case.
“GYS1-specific inhibitor” refers to any agent capable of specifically inhibiting GYS1 expression or activity at the molecular level. For example, GYS1-specific inhibitors include nucleic acids (including antisense compounds), peptides, antibodies, small molecules, and other agents capable of inhibiting the expression or activity of GYS1.
“Hybridization” means annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.
“Immediately adjacent” means there are no intervening elements between the immediately adjacent elements of the same kind (e.g. no intervening nucleobases between the immediately adjacent nucleobases).
“Individual” means a human or non-human animal selected for treatment or therapy.
“Inhibiting the expression or activity” refers to a reduction or blockade of the expression or activity relative to the expression of activity in an untreated or control sample and does not necessarily indicate a total elimination of expression or activity.
“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.
“Intraperitoneal administration” means administration through infusion or injection into the peritoneum.
“Intravenous administration” means administration into a vein.
“Lafora bodies” are massive neurotoxic inclusions formed as a result of mis-structuring of glycogen and its precipitation and accumulation to form polyglucosan.
“Lafora disease” (LD) means the severe and fatal form of adolescence-onset epilepsy resulting from accumulation of Lafora bodies in neurons, muscle, and other tissues.
“Lengthened oligonucleotides” are those that have one or more additional nucleosides relative to an oligonucleotide disclosed herein, e.g. a parent oligonucleotide.
“Linked nucleosides” means adjacent nucleosides linked together by an internucleoside linkage.
“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.
“Modulating” refers to changing or adjusting a feature in a cell, tissue, organ or organism. For example, modulating GYS1 can mean to increase or decrease the level of GYS1 in a cell, tissue, organ or organism. A “modulator” effects the change in the cell, tissue, organ or organism. For example, a compound can be a modulator of GYS1 that decreases the amount of GYS1 in a cell, tissue, organ or organism.
“MOE” means methoxyethyl.
“Monomer” refers to a single unit of an oligomer. Monomers include, but are not limited to, nucleosides and nucleotides.
“Motif” means the pattern of unmodified and/or modified sugar moieties, nucleobases, and/or internucleoside linkages, in an oligonucleotide.
“Myoclonus” means episodes of repeated stereotypic, involuntary muscle jerking or twitching that can affect part of the body or the entire body for variable durations.
“Natural” or “naturally occurring” means found in nature.
“Non-bicyclic modified sugar” or “non-bicyclic modified sugar moiety” means a modified sugar moiety that comprises a modification, such as a substituent, that does not form a bridge between two atoms of the sugar to form a second ring.
“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.
“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.
“Nucleobase sequence” means the order of contiguous nucleobases in a nucleic acid or oligonucleotide independent of any sugar or internucleoside linkage.
“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.
“Oligomeric compound” means a compound comprising a single oligonucleotide and optionally one or more additional features, such as a conjugate group or terminal group.
“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.
“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.
“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.
“Pharmaceutically acceptable carrier or diluent” means any substance suitable for use in administering to an animal. For example, a pharmaceutically acceptable carrier can be a sterile aqueous solution, such as PBS or water-for-injection.
“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.
“Pharmaceutical agent” means a compound that provides a therapeutic benefit when administered to an individual.
“Pharmaceutical composition” means a mixture of substances suitable for administering to an individual. For example, a pharmaceutical composition may comprise one or more compounds or salt thereof and a sterile aqueous solution.
“Phosphorothioate linkage” means a modified phosphate linkage in which one of the non-bridging oxygen atoms is replaced with a sulfur atom. A phosphorothioate internucleoside linkage is a modified internucleoside linkage.
“Phosphorus moiety” means a group of atoms comprising a phosphorus atom. In certain embodiments, a phosphorus moiety comprises a mono-, di-, or tri-phosphate, or phosphorothioate.
“Pompe disease” also called glycogen storage disease type II, is a neuromuscular disorder caused by buildup of glycogen in the body's cells. Pompe disease is a single disease continuum with variable rates of disease progression and different ages of onset. The first symptoms can occur at any age from birth to late adulthood.
“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.
“Prevent” refers to delaying or forestalling the onset, development or progression of a disease, disorder, or condition for a period of time from minutes to indefinitely.
“Prodrug” means a compound in a form outside the body which, when administered to an individual, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.
“Reduce” means to bring down to a smaller extent, size, amount, or number.
“RefSeq No.” is a unique combination of letters and numbers assigned to a sequence to indicate the sequence is for a particular target transcript (e.g., target gene). Such sequence and information about the target gene (collectively, the gene record) can be found in a genetic sequence database. Genetic sequence databases include the NCBI Reference Sequence database, GenBank, the European Nucleotide Archive, and the DNA Data Bank of Japan (the latter three forming the International Nucleotide Sequence Database Collaboration or INSDC).
“Region” is defined as a portion of the target nucleic acid having at least one identifiable structure, function, or characteristic.
“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.
“Segments” are defined as smaller or sub-portions of regions within a nucleic acid.
“Seizures” are a symptom of many different disorders and conditions that can affect the brain. “Seizures” are typically caused by disruptions in the electric communication between neurons in the brain, resulting from a brain injury or a disease or disorder. Seizures can take on different forms and affect different people in different ways. Common physical changes that may occur during a seizure are difficulty talking, inability to swallow, drooling, repeated blinking of the eyes, staring, lack of movement of muscle tone, slumping tremors, twitching, or jerking movements, rigid or tense muscles, repeated non-purposeful movements, called automatisms, involving the face, arms, or legs, convulsions, loss of control of urine or stool, sweating, change in skin color (paleness or flushing), dilation of pupils, biting of tongue, difficulty breathing, heart palpitations. In some embodiments, seizures are mild. In other embodiments, seizures are completely disabling or may result in death. Abnormal brain activity can often be documented by abnormal findings on an electroencephalogram (EEG).
“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.
“Single-stranded” in reference to a compound means the compound has only one oligonucleotide. “Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.
“Sites,” are defined as unique nucleobase positions within a target nucleic acid.
“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.
“Specifically inhibit” a target nucleic acid means to reduce or block expression of the target nucleic acid while exhibiting fewer, minimal, or no effects on non-target nucleic acids reduction and does not necessarily indicate a total elimination of the target nucleic acid's expression.
“Standard cell assay” means assay(s) described in the Examples and reasonable variations thereof.
“Standard in vivo experiment” means the procedure(s) described in the Example(s) and reasonable variations thereof.
“Sugar moiety” means an unmodified sugar moiety or a modified sugar moiety. “Unmodified sugar moiety” or “unmodified sugar” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.
“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.
“Subcutaneous administration” means administration just below the skin.
“Target gene” refers to a gene encoding a target.
“Targeting” means specific hybridization of a compound that to a target nucleic acid in order to induce a desired effect.
“Target nucleic acid,” “target RNA,” “target RNA transcript” and “nucleic acid target” all mean a nucleic acid capable of being targeted by compounds described herein.
“Target region” means a portion of a target nucleic acid to which one or more compounds is targeted.
“Target segment” means the sequence of nucleotides of a target nucleic acid to which a compound described herein is targeted. “5′ target site” refers to the 5′-most nucleotide of a target segment. “3′ target site” refers to the 3′-most nucleotide of a target segment.
“Terminal group” means a chemical group or group of atoms that is covalently linked to a terminus of an oligonucleotide.
“Therapeutically effective amount” means an amount of a compound, pharmaceutical agent, or composition that provides a therapeutic benefit to an individual.
“Treat” refers to administering a compound or pharmaceutical composition to an animal in order to effect an alteration or improvement of a disease, disorder, or condition in the animal.

### Certain Embodiments

Certain embodiments provide methods, compounds, and compositions for modulating a glycogen storage disease or a polyglucosan disorder or a symptom thereof, in an individual by administering the compound or composition to the individual, wherein the compound or composition comprises a GYS1 modulator. Modulation of GYS1 can lead to a decrease of GYS1 level or expression in order to reduce glycogen synthesis and aggregation in order to treat, prevent, ameliorate or delay a glycogen storage disease, or a symptom thereof. In certain embodiments, the GYS1 modulator is a GYS1-specific inhibitor. In certain embodiments, GYS1-specific inhibitors are nucleic acids (including antisense compounds), peptides, antibodies, small molecules, and other agents capable of inhibiting the expression or activity of GYS1. In certain embodiments, the individual is human.

Certain embodiments disclosed herein provide compounds or compositions comprising a GYS1 modulator. Such compounds or compositions are useful to treat, prevent, amelioarate, or delay the onset of a glycogen storage disease, or a symptom thereof. In certain embodiments, the compound comprises a GYS1-specific inhibitor. In certain embodiments, the GYS1-specific inhibitor is a nucleic acid, polypeptide, antibody, small molecules, or other agent capable of inhibiting the expression or activity of GYS1. In certain embodiments, a GYS1-specific inhibitor is a nucleic acid targeting GYS1. In certain embodiments, the nucleic acid is single stranded. In certain embodiments, the nucleic acid is double stranded. In certain embodiments, the compound or composition comprises an antisense compound. In any of the foregoing embodiments, the compound or composition comprises an oligomeric compound. In certain embodiments, the compound or composition comprises an oligonucleotide targeting GYS1. In certain embodiments, the oligonucleotide is single stranded. In certain embodiments, the compound comprises deoxyribonucleotides. In certain embodiments, the compound comprises ribonucleotides and is double-stranded. In certain embodiments, the oligonucleotide is a modified oligonucleotide. In certain embodiments, the modified oligonucleotide is single stranded.

In any of the foregoing embodiments, the compound can comprise a modified oligonucleotide 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides in length.

In certain embodiments, at least one internucleoside linkage of said modified oligonucleotide is a modified internucleoside linkage. In certain embodiments, at least one internucleoside linkage is a phosphorothioate internucleoside linkage. In certain embodiments, the internucleoside linkages are phosphorothioate linkages and phosphate ester linkages.

In certain embodiments, any of the foregoing oligonucleotides comprises at least one modified sugar. In certain embodiments, at least one modified sugar comprises a 2′-O-methoxyethyl group. In certain embodiments, at least one modified sugar is a bicyclic sugar, such as a 4′-CH(CH₃)—O-2′ group, a 4′-CH₂—O-2′ group, or a 4′-(CH₂)₂—O-2′ group.

In certain embodiments, at least one nucleoside of said modified oligonucleotide comprises a modified nucleobase. In certain embodiments, the modified nucleobase is a 5-methylcytosine.

In certain embodiments, a compound or composition comprises a modified oligonucleotide comprising: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar. In certain embodiments, at least one internucleoside linkage is a phosphorothioate linkage. In certain embodiments, and at least one cytosine is a 5-methylcytosine.

In certain embodiments, a compound comprises a modified oligonucleotide 12 to 80 linked nucleosides in length and having a nucleobase sequence comprising the nucleobase sequence of any one of SEQ ID Nos: 10-76. In certain embodiments, the compound is an antisense compound or oligomeric compound. In certain embodiments, the compound is single-stranded. In certain embodiments, the compound is double-stranded. In certain embodiments, the modified oligonucleotide is 12 to 30 linked nucleosides in length.

In certain embodiments, the compounds or compositions disclosed herein further comprise a pharmaceutically acceptable carrier or diluent.

In certain embodiments, the compound or composition is co-administered with a second agent. In certain embodiments, the compound and the second agent are administered concomitantly.

In certain embodiments, compounds and compositions described herein targeting GYS1 can be used in methods of inhibiting expression of GYS1 in a cell. In certain embodiments, compounds and compositions described herein targeting GYS1 can be used in methods of treating, preventing, delaying, or ameliorating a glycogen storage disease or a polyglucosan disorder, including, but not limited to, Lafora disease, adult polyglucosan body disease, and Pompe disease.

### Certain Indications

Certain embodiments provided herein relate to methods of inhibiting GYS1 expression or activity, which can be useful for treating, preventing, or ameliorating a disease associated with GYS1 in an individual, by administration of a compound or composition that targets GYS1. In certain embodiments, such a compound or composition comprises a GYS1-specific inhibitor. In certain embodiments, the compound comprises an antisense compound or an oligomeric compound targeted to GYS1. In certain embodiments, the compound comprises a modified oligonucleotide targeted to GYS1.

In certain embodiments, a method of inhibiting expression or activity of GYS1 in a cell comprises contacting the cell with a compound or composition comprising a GYS1-specific inhibitor, thereby inhibiting the expression or activity of GYS1 in the cell. In certain embodiments, the cell is a neuron. In certain embodiments, the cell is a hepatocyte. In certain embodiments, the cell is a skeletal muscle cell. In certain embodiments, the cell is a cardiac muscle cell. In certain embodiments, the cell is in the brain tissue, in the liver, in the heart, or in the skeletal muscle. In certain embodiments, the cell is in the brain, liver, heart, or skeletal muscle tissue of an individual who has, or is at risk of having a disease, disorder, condition, symptom, or physiological marker associated with a glycogen storage disease or a polyglucosan disorder. In certain embodiments, the polyglucosan disease or disorder is Lafora disease. In certain embodiments, the polyglucosan disease or disorder is adult polyglucosan body disease. In certain embodiments, the disease or disorder is Andersen's disease. In certain embodiments, the polyglucosan disease or disorder is Pompe disease. In certain embodiments, the GYS1-specific inhibitor is a nucleic acid, peptide, antibody, small molecule or other agent capable of inhibiting the expression or activity of the GYS1. In certain embodiments, the GYS1-specific inhibitor is an antisense compound or an oligomeric compound targeted to GYS1. In certain embodiments, the GYS1-specific inhibitor is oligonucleotide targeted to GYS1. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded.

In certain embodiments, a method of treating, preventing, delaying the onset, slowing the progression, or ameliorating one or more disease, disorders, conditions, symptoms, or physiological markers associated with GYS1 comprises administering to the individual a compound or composition comprising a GYS1-specific inhibitor. In certain embodiments, a method of treating, preventing, delaying the onset, slowing the progression, or ameliorating a disease, disorder, condition, symptom, or physiological marker associated with a with a glycogen storage disease or a polyglucosan disease or disorder in an individual comprises administering to the individual a compound or composition comprising a GYS1-specific inhibitor, thereby treating, preventing, delaying the onset, slowing the progression, or ameliorating the disease. In certain embodiments, the individual is identified as having, or at risk of having, the disease, disorder, condition, symptom or physiological marker. In certain embodiments, the polyglucosan disease or disorder is Lafora disease. In certain embodiments, the polyglucosan disease or disorder is adult polyglucosan body disease. In certain embodiments, the disease or disorder is Andersen's disease. In certain embodiments, the polyglucosan disease or disorder is Pompe disease. In certain embodiments, the GYS1-specific inhibitor is administered to the individual parenterally. In certain embodiments, the parenteral administration is intracerebroventricular administration. In certain embodiments, the parenteral administration is intrathecal administration. In certain embodiments, the parenteral administration is subcutaneous administration. In certain embodiments, the individual is human. In certain embodiments, the GYS1-specific inhibitor is a nucleic acid, peptide, antibody, small molecule or other agent capable of inhibiting the expression or activity of GYS1. In certain embodiments, the GYS1-specific inhibitor comprises an antisense compound or an oligomeric compound targeted to GYS1. In certain embodiments, the GYS1-specific inhibitor is an oligonucleotide targeted to GYS1. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded.

In certain embodiments, a method of reducing seizures, decreasing myoclonus or muscle spasms, alleviating difficulty in walking (peripheral neuropathy), spasticity, reducing, preventing the onset of, or treating dementia, alleviating difficulties in speech, reducing or preventing the onset of visual hallucinations, treating, reducing or preventing the onset of progressive neurologic degeneration, treating, reducing, or preventing the onset of damage to nerves that control bladder function, lessening hypotonia, improving muscle tone, reducing or preventing the onset of an enlarged liver, reducing or preventing the onset of heart defects, reducing or preventing the accumulation of polyglucosan bodies in a cell. reducing or preventing the accumulation of lafora bodies in a cell, reducing glycogen accumulation in a cell, improving or preventing cognitive deterioration, and reducing ataxia, or a combination thereof, in an individual comprises administering to the individual a compound or composition comprising a GYS1-specific inhibitor. In certain embodiments, the cell is a neuron. In certain embodiments, the cell is a hepatocyte. In certain embodiments, the cell is a skeletal muscle cell. In certain embodiments, the cell is a cardiac muscle cell. In certain embodiments, administering the compound or composition reduces seizures in the individual. In certain embodiments, administering the compound or composition decreases myoclonus or muscle spasms in the individual. In certain embodiments, administering the compound or composition alleviates difficulty in walking in the individual. In certain embodiments, administering the compound or composition alleviates peripheral neuropathy in the individual. In certain embodiments, administering the compound or composition alleviates spasticity in the individual. In certain embodiments, administering the compound or composition reduces, prevents the onset of, or treats dementia in the individual. In certain embodiments, administering the compound or composition alleviates difficulties in speech in the individual. In certain embodiments, administering the compound or composition reduces or prevents the onset of visual hallucinations in the individual. In certain embodiments, administering the compound or composition treats, reduces or prevents the onset of progressive neurologic degeneration in the individual. In certain embodiments, administering the compound or composition treats, reduces or prevents the onset of damage to the nerves that control bladder function in the individual. In certain embodiments, administering the compound or composition treats, reduces or prevents the onset of hypotonia in the individual. In certain embodiments, administering the compound or composition improves muscle tone in the individual. In certain embodiments, administering the compound or composition treats, reduces or prevents the onset of liver enlargement in the individual. In certain embodiments, administering the compound or composition treats, reduces or prevents the onset of heart defects in the individual. In certain embodiments, administering the compound or composition treats, reduces or prevents the onset of polyglucosan bodies in a cell in the individual. In certain embodiments, administering the compound or composition treats, reduces or prevents the onset of lafora bodies in a cell in the individual. In certain embodiments, administering the compound or composition treats, reduces or prevents the onset of glycogen accumulation in a cell in the individual. In certain embodiments, the cell is a neuron. In certain embodiments, the cell is a hepatocyte. In certain embodiments, the cell is a skeletal muscle cell. In certain embodiments, the cell is a cardiac muscle cell. In certain embodiments, administering the compound or composition improves or prevents cognitive deterioration. In certain embodiments, administering the compound or composition treats, reduces ataxia in the individual. In certain embodiments, the individual is identified as having, or at risk of having a disease, disorder, condition, symptom, or physiological marker associated with a glycogen storage disease or a polyglucosan disease or disorder. In certain embodiments, the polyglucosan disease or disorder is Lafora disease. In certain embodiments, the polyglucosan disease or disorder is adult polyglucosan body disease. In certain embodiments, the disease or disorder is Andersen's disease. In certain embodiments, the polyglucosan disease or disorder is Pompe disease. In certain embodiments, the GYS1-specific inhibitor is administered to the individual parenterally. In certain embodiments, the parenteral administration is intracerebroventricular administration. In certain embodiments, the parenteral administration is intrathecal administration. In certain embodiments, the parenteral administration is subcutaneous administration. In certain embodiments, the individual is human. In certain embodiments, the GYS1-specific inhibitor is a nucleic acid, peptide, antibody, small molecule or other agent capable of inhibiting the expression or activity of the GYS1. In certain embodiments, the GYS1-specific inhibitor is an antisense compound or an oligomeric compound targeted to GYS1. In certain embodiments, the GYS1-specific inhibitor is oligonucleotide targeted to GYS1. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded.

In certain embodiments, administering the compound or composition disclosed herein decreases seizures, decreases myoclonus or muscle spasms, alleviates difficulty in walking, alleviates spasticity, reduces, prevents the onset of or treats dementia, alleviates difficulties in speech, reduces or prevents the onset of visual hallucinations, treats, reduces or prevents the onset of progressive neurologic degeneration, treating, reducing, or preventing the onset of damage to nerves that control bladder function, lessening hypotonia, improving muscle tone, reducing or preventing the onset of an enlarged liver, reducing or preventing the onset of heart defects, reduces polyglucosan bodies in a cell, reduces lafora bodies in a cell, reduces glycogen accumulation in a cell, improves cognitive deterioration, and reduces ataxia, or a combination thereof. In certain embodiments, seizures were independently reduced by at least 5%, at least 10%, at least 20%, at least 30%, at least 35%, at least 40%, at least 45% or at least 50%. In certain embodiments, myoclonus or muscle spasms were independently reduced by at least 5%, at least 10%, at least 20%, at least 30%, at least 35%, at least 40%, at least 45% or at least 50%. In certain embodiments, difficulty in walking was independently alleviated by at least 5%, at least 10%, at least 20%, at least 30%, at least 35%, at least 40%, at least 45% or at least 50%. In certain embodiments, spasticity was independently reduced by at least 5%, at least 10%, at least 20%, at least 30%, at least 35%, at least 40%, at least 45% or at least 50%. In certain embodiments, difficulty in speech was independently alleviated by at least 5%, at least 10%, at least 20%, at least 30%, at least 35%, at least 40%, at least 45% or at least 50%. In certain embodiments, visual hallucinations were independently reduced by at least 5%, at least 10%, at least 20%, at least 30%, at least 35%, at least 40%, at least 45% or at least 50%. In certain embodiments, progressive neurologic degeneration was independently reduced by at least 5%, at least 10%, at least 20%, at least 30%, at least 35%, at least 40%, at least 45% or at least 50%. In certain embodiments, dementia progression was independently reduced by at least 5%, at least 10%, at least 20%, at least 30%, at least 35%, at least 40%, at least 45% or at least 50%. In certain embodiments, nerve damage of bladder function independently reduced by at least 5%, at least 10%, at least 20%, at least 30%, at least 35%, at least 40%, at least 45% or at least 50%. In certain embodiments, hypotonia was independently reduced by at least 5%, at least 10%, at least 20%, at least 30%, at least 35%, at least 40%, at least 45% or at least 50%. In certain embodiments, liver enlargement was independently reduced by at least 5%, at least 10%, at least 20%, at least 30%, at least 35%, at least 40%, at least 45% or at least 50%. In certain embodiments, heart defects were independently reduced by at least 5%, at least 10%, at least 20%, at least 30%, at least 35%, at least 40%, at least 45% or at least 50%. In certain embodiments, polyglucosan bodies in a cell were independently reduced by at least 5%, at least 10%, at least 20%, at least 30%, at least 35%, at least 40%, at least 45% or at least 50%. In certain embodiments, lafora bodies in a cell were independently reduced by at least 5%, at least 10%, at least 20%, at least 30%, at least 35%, at least 40%, at least 45% or at least 50%. In certain embodiments, glycogen accumulation in a cell were independently reduced by at least 5%, at least 10%, at least 20%, at least 30%, at least 35%, at least 40%, at least 45% or at least 50%. In certain embodiments, cognitive deterioration was reduced by at least 5%, at least 10%, at least 20%, at least 30%, at least 35%, at least 40%, at least 45% or at least 50%. In certain embodiments, ataxia was independently reduced by at least 5%, at least 10%, at least 20%, at least 30%, at least 35%, at least 40%, at least 45% or at least 50%. In certain embodiments, the cell is a neuron. In certain embodiments, the cell is a hepatocyte. In certain embodiments, the cell is a skeletal muscle cell. In certain embodiments, the cell is a cardiac muscle cell.

Certain embodiments provide compounds and compositions described herein for use in therapy. Certain embodiments are drawn to a compound or composition comprising a GYS1-specific inhibitor for use in treating, preventing, delaying the onset, slowing the progression, or ameliorating one or more diseases, disorders, conditions, symptoms, or physiological markers associated with GYS1. Certain embodiments are drawn to a compound or composition for use in treating, preventing, delaying the onset, slowing the progression, or ameliorating a glycogen storage disease or a polyglucosan disease or disorder, or a symptom or physiological marker thereof In certain embodiments, the polyglucosan disease or disorder is Lafora disease. In certain embodiments, the polyglucosan disease or disorder is adult polyglucosan body disease. In certain embodiments, the disease or disorder is Andersen's disease.

In certain embodiments, the polyglucosan disease or disorder is Pompe disease. In certain embodiments, the GYS1-specific inhibitor is a nucleic acid, peptide, antibody, small molecule or other agent capable of inhibiting the expression or activity of the GYS1. In certain embodiments, the GYS1-specific inhibitor is an antisense compound or an oligomeric compound targeted to GYS1. In certain embodiments, the GYS1-specific inhibitor is oligonucleotide targeted to GYS1. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded.

Certain embodiments are drawn to a compound or composition comprising a GYS1-specific inhibitor for use in reducing seizures, decreasing myoclonus or muscle spasms, alleviating difficulty in walking, reducing, preventing the onset of, or treating dementia, alleviating difficulties in speech, reducing or preventing the onset of visual hallucinations, treating, reducing or preventing the onset of progressive neurologic degeneration, treating, reducing, or preventing the onset of damage to nerves that control bladder function, lessening hypotonia, improving muscle tone, reducing or preventing the onset of an enlarged liver, reducing or preventing the onset of heart defects, reducing or preventing the accumulation of polyglucosan bodies in a cell. reducing or preventing the accumulation of lafora bodies in a cell, reducing glycogen accumulation in a cell, improving or preventing cognitive deterioration, and reducing ataxia, or a combination thereof, in an individual. In certain embodiments, administering the compound or composition reduces seizures in the individual. In certain embodiments, administering the compound or composition decreases myoclonus or muscle spasms in the individual. In certain embodiments, administering the compound or composition alleviates difficulty in walking in the individual. In certain embodiments, administering the compound or composition reduces, prevents the onset of, or treats dementia in the individual. In certain embodiments, administering the compound or composition alleviates difficulties in speech in the individual. In certain embodiments, administering the compound or composition reduces or prevents the onset of visual hallucinations in the individual. In certain embodiments, administering the compound or composition treats, reduces or prevents the onset of progressive neurologic degeneration in the individual. In certain embodiments, administering the compound or composition treats, reduces, or prevents the onset of damage to nerves that control bladder function in the individual. In certain embodiments, administering the compound or composition treats, reduces, or prevents hypotonia in the individual. In certain embodiments, administering the compound or composition improves muscle tone in the individual. In certain embodiments, administering the compound or composition reduces or prevents the onset of an enlarged liver in the individual. In certain embodiments, administering the compound or composition reduces or prevents the onset of heart defects in the individual. In certain embodiments, administering the compound or composition treats, reduces or prevents the onset of polyglucosan bodies in a cell in the individual. In certain embodiments, administering the compound or composition treats, reduces or prevents the onset of lafora bodies in a cell in the individual. In certain embodiments, administering the compound or composition treats, reduces or prevents the onset of glycogen accumulation in a cell in the individual. In certain embodiments, the cell is a neuron. In certain embodiments, the cell is a hepatocyte. In certain embodiments, the cell is a skeletal muscle cell. In certain embodiments, the cell is a cardiac muscle cell. In certain embodiments, administering the compound or composition improves or prevents cognitive deterioration. In certain embodiments, administering the compound or composition treats, reduces ataxia in the individual. In certain embodiments, the individual is identified as having, or at risk of having a disease, disorder, condition, symptom, or physiological marker associated with a glycogen storage disease or a polyglucosan disease or disorder. In certain embodiments, the polyglucosan disease or disorder is Lafora disease. In certain embodiments, the polyglucosan disease or disorder is adult polyglucosan body disease. In certain embodiments, the disease or disorder is Andersen's disease. In certain embodiments, the polyglucosan disease or disorder is Pompe disease. In certain embodiments, the individual is human. In certain embodiments, the GYS1-specific inhibitor is a nucleic acid, peptide, antibody, small molecule or other agent capable of inhibiting the expression or activity of the GYS1. In certain embodiments, the GYS1-specific inhibitor is an antisense compound or an oligomeric compound targeted to GYS1. In certain embodiments, the GYS1-specific inhibitor is oligonucleotide targeted to GYS1. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound comprising a modified oligonucleotide can be single-stranded. In certain embodiments, the compound comprising a modified oligonucleotide can be double-stranded.

Certain embodiments are drawn to the use of compounds or compositions described herein for the manufacture or preparation of a medicament for therapy. Certain embodiments are drawn to the use of a compound or composition as described herein in the manufacture or preparation of a medicament for treating, preventing, delaying the onset, slowing progression, or ameliorating one or more diseases, disorders, conditions, symptoms, or physiological markers associated with GYS1. In certain embodiments, the compound or composition as described herein is used in the manufacture or preparation of a medicament for treating, ameliorating, delaying or preventing a glycogen storage disease or a polyglucosan disease or disorder. In certain embodiments, the polyglucosan disease or disorder is Lafora disease. In certain embodiments, the polyglucosan disease or disorder is adult polyglucosan body disease. In certain embodiments, the disease or disorder is Andersen's disease. In certain embodiments, the polyglucosan disease or disorder is Pompe disease. In certain embodiments, the compound or composition comprises a nucleic acid, peptide, antibody, small molecule or other agent capable of inhibiting the expression or activity of GYS1. In certain embodiments, the compound or composition comprises an antisense compound or an oligomeric compound targeted to GYS1. In certain embodiments, the compound or composition comprises an oligonucleotide targeted to GYS1. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound or composition comprising a modified oligonucleotide can be single-stranded. In certain embodiments, the compound or composition comprising a modified oligonucleotide can be double-stranded.

Certain embodiments are drawn to the use of a compound or composition for the manufacture or preparation of a medicament for reducing seizures, decreasing myoclonus or muscle spasms, alleviating difficulty in walking, reducing, preventing the onset of, or treating dementia, alleviating difficulties in speech, reducing or preventing the onset of visual hallucinations, treating, reducing or preventing the onset of progressive neurologic degeneration, treating, reducing, or preventing the onset of damage to nerves that control bladder function, lessening hypotonia, improving muscle tone, reducing or preventing the onset of an enlarged liver, reducing or preventing the onset of heart defects, reducing or preventing the accumulation of polyglucosan bodies in a cell. reducing or preventing the accumulation of lafora bodies in a cell, reducing glycogen accumulation in a cell, improving or preventing cognitive deterioration, and reducing ataxia, or a combination thereof, in an individual having or at risk of having a glycogen storage disease or a polyglucosan disease or disorder. In certain embodiments, the cell is a neuron. In certain embodiments, the cell is a hepatocyte. In certain embodiments, the cell is a skeletal muscle cell. In certain embodiments, the cell is a cardiac muscle cell. Certain embodiments are drawn to use of a compound or composition in the manufacture or preparation of a medicament for reducing seizures in the individual. Certain embodiments are drawn to use of a compound or composition in the manufacture or preparation of a medicament for decreasing myoclonus or muscle spasms in the individual. Certain embodiments are drawn to use of a compound or composition in the manufacture or preparation of a medicament for alleviating difficulty in walking in the individual. Certain embodiments are drawn to use of a compound or composition in the manufacture or preparation of a medicament for reducing, preventing the onset of, or treating dementia in the individual. Certain embodiments are drawn to use of a compound or composition in the manufacture or preparation of a medicament alleviating difficulties in speech in the individual. Certain embodiments are drawn to use of a compound or composition in the manufacture or preparation of a medicament reducing or preventing the onset of visual hallucinations in the individual. Certain embodiments are drawn to use of a compound or composition in the manufacture or preparation of a medicament treating, reducing or preventing the onset of progressive neurologic degeneration in the individual. Certain embodiments are drawn to the use of a compound or composition in the manufacture or preparation of a medicament for treating, reducing, or preventing the onset of damage to nerves that control bladder function in the individual. Certain embodiments are drawn to the use of a compound or composition in the manufacture or preparation of a medicament for treating, reducing, or preventing hypotonia in the individual. Certain embodiments are drawn to the use of a compound or composition in the manufacture or preparation of a medicament for improving muscle tone in the individual. Certain embodiments are drawn to the use of a compound or composition in the manufacture or preparation of a medicament for treating, reducing, or preventing the onset of an enlarged liver in the individual. Certain embodiments are drawn to the use of a compound or composition in the manufacture or preparation of a medicament for treating, reducing, or preventing the onset of heart defects in the individual. Certain embodiments are drawn to the use of a compound or composition in the manufacture or preparation of a medicament for treating, reducing, or preventing the onset of polyglucosan bodies in a cell in the individual. Certain embodiments are drawn to the use of a compound or composition in the manufacture or preparation of a medicament for treating, reducing, or preventing the onset of lafora bodies in the individual. Certain embodiments are drawn to the use of a compound or composition in the manufacture or preparation of a medicament for treating, reducing, or preventing the onset of glycogen accumulation in the individual. Certain embodiments are drawn to use of a compound or composition in the manufacture or preparation of a medicament reducing ataxia in the individual. In certain embodiments, the cell is a neuron. In certain embodiments, the cell is a hepatocyte. In certain embodiments, the cell is a skeletal muscle cell. In certain embodiments, the cell is a cardiac muscle cell. In certain embodiments, the compound or composition comprises a nucleic acid, peptide, antibody, small molecule or other agent capable of inhibiting the expression or activity of the GYS1. In certain embodiments, the compound or composition comprises an antisense compound or an oligomeric compound targeted to GYS1. In certain embodiments, the compound or composition comprises an oligonucleotide targeted to GYS1. In certain embodiments, the compound or composition comprises a modified oligonucleotide 8 to 80 linked nucleosides in length. In certain embodiments, the compound or composition comprises a modified oligonucleotide 10 to 30 linked nucleosides in length. In certain embodiments, the compound or composition comprising a modified oligonucleotide can be single-stranded. In certain embodiments, the compound or composition comprising a modified oligonucleotide can be double-stranded.

In any of the foregoing methods or uses, the compound or composition can comprise an antisense compound targeted to GYS1. In certain embodiments, the compound comprises an oligonucleotide, for example an oligonucleotide consisting of 8 to 80 linked nucleosides, 10 to 30 linked nucleosides, 12 to 30 linked nucleosides, or 20 linked nucleosides. In certain embodiments, the oligonucleotide comprises at least one modified internucleoside linkage, at least one modified sugar and/or at least one modified nucleobase. In certain embodiments, the modified internucleoside linkage is a phosphorothioate internucleoside linkage, the modified sugar is a bicyclic sugar or a 2′-O-methoxyethyl, and the modified nucleobase is a 5-methylcytosine. In certain embodiments, the modified oligonucleotide comprises a gap segment consisting of linked deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each nucleoside of each wing segment comprises a modified sugar. In certain embodiments, the compound can comprise a modified oligonucleotide 12 to 80 linked nucleosides in length and having a nucleobase sequence comprising the nucleobase sequences of any one of SEQ ID NOs: 10-76. In certain embodiments, the compound is an antisense compound or oligomeric compound. In certain embodiments, the compound is single-stranded. In certain embodiments, the compound is double-stranded. In certain embodiments, the modified oligonucleotide is 12 to 30 linked nucleosides in length. In certain embodiments, the compounds or compositions disclosed herein further comprise a pharmaceutically acceptable carrier or diluent.

In any of the foregoing methods or uses, the compound or composition comprises or consists of a modified oligonucleotide 12 to 30 linked nucleosides in length, wherein the modified oligonucleotide comprises:
a gap segment consisting of linked 2′-deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar.

In any of the foregoing methods or uses, the compound or composition can be administered parenterally. For example, in certain embodiments the compound or composition can be administered through injection or infusion. Parenteral administration includes subcutaneous administration, intravenous administration, intramuscular administration, intraarterial administration, intraperitoneal administration, or intracranial administration. In certain embodiments, the compound or composition is co-administered with a second agent. In certain embodiments, the compound or composition and the second agent are administered concomitantly.

### Certain Compounds

In certain embodiments, compounds described herein are antisense compounds. In certain embodiments, the antisense compound comprises or consists of an oligomeric compound. In certain embodiments, the oligomeric compound comprises a modified oligonucleotide. In certain embodiments, the modified oligonucleotide has a nucleobase sequence complementary to that of a target nucleic acid.

In certain embodiments, a compound described herein comprises or consists of a modified oligonucleotide. In certain embodiments, the modified oligonucleotide has a nucleobase sequence complementary to that of a target nucleic acid.

In certain embodiments, a compound or antisense compound is single-stranded. Such a single-stranded compound or antisense compound comprises or consists of an oligomeric compound. In certain embodiments, such an oligomeric compound comprises or consists of an oligonucleotide. In certain embodiments, the oligonucleotide is an antisense oligonucleotide. In certain embodiments, the oligonucleotide is modified. In certain embodiments, the oligonucleotide of a single-stranded antisense compound or oligomeric compound comprises a self-complementary nucleobase sequence.

In certain embodiments, compounds are double-stranded. Such double-stranded compounds comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide. In such embodiments, the thymine nucleobase in the modified oligonucleotide is replaced by a uracil nucleobase. In certain embodiments, compound comprises a conjugate group. In certain embodiments, each modified oligonucleotide is 12-30 linked nucleosides in length.

In certain embodiments, compounds are double-stranded. Such double-stranded compounds comprise a first oligomeric compound having a region complementary to a target nucleic acid and a second oligomeric compound having a region complementary to the first oligomeric compound. The first oligomeric compound of such double stranded compounds typically comprises or consists of a modified oligonucleotide. The oligonucleotide of the second oligomeric compound of such double-stranded compound may be modified or unmodified. The oligomeric compounds of double-stranded compounds may include non-complementary overhanging nucleosides.

Examples of single-stranded and double-stranded compounds include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

In certain embodiments, a compound described herein comprises an oligonucleotide 10 to 30 linked subunits in length. In certain embodiments, compound described herein comprises an oligonucleotide is 12 to 30 linked subunits in length. In certain embodiments, compound described herein comprises an oligonucleotide is 12 to 22 linked subunits in length. In certain embodiments, compound described herein comprises an oligonucleotide is 14 to 30 linked subunits in length. In certain embodiments, compound described herein comprises an oligonucleotide is 14 to 20 linked subunits in length. In certain embodiments, compound described herein comprises an oligonucleotide is 15 to 30 linked subunits in length. In certain embodiments, compound described herein comprises an oligonucleotide is 15 to 20 linked subunits in length. In certain embodiments, compound described herein comprises an oligonucleotide is 16 to 30 linked subunits in length. In certain embodiments, compound described herein comprises an oligonucleotide is 16 to 20 linked subunits in length. In certain embodiments, compound described herein comprises an oligonucleotide is 17 to 30 linked subunits in length. In certain embodiments, compound described herein comprises an oligonucleotide is 17 to 20 linked subunits in length. In certain embodiments, compound described herein comprises an oligonucleotide is 18 to 30 linked subunits in length. In certain embodiments, compound described herein comprises an oligonucleotide is 18 to 21 linked subunits in length. In certain embodiments, compound described herein comprises an oligonucleotide is 18 to 20 linked subunits in length. In certain embodiments, compound described herein comprises an oligonucleotide is 20 to 30 linked subunits in length. In other words, such oligonucleotides are from 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the linked subunits are nucleotides, nucleosides, or nucleobases.

In certain embodiments, compounds may be shortened or truncated. For example, a single subunit may be deleted from the 5′ end (5′ truncation), or alternatively from the 3′ end (3′ truncation). A shortened or truncated compound targeted to a GYS1 nucleic acid may have two subunits deleted from the 5′ end, or alternatively may have two subunits deleted from the 3′ end, of the compound. Alternatively, the deleted nucleosides may be dispersed throughout the compound.

When a single additional subunit is present in a lengthened compound, the additional subunit may be located at the 5′ or 3′ end of the compound. When two or more additional subunits are present, the added subunits may be adjacent to each other, for example, in a compound having two subunits added to the 5′ end (5′ addition), or alternatively to the 3′ end (3′ addition), of the compound. Alternatively, the added subunits may be dispersed throughout the compound.

It is possible to increase or decrease the length of a compound, such as an oligonucleotide, and/or introduce mismatch bases without eliminating activity (Woolf et al. (Proc. Natl. Acad. Sci. USA 89:7305-7309, 1992; Gautschi et al. J. Natl. Cancer Inst. 93:463-471, March 2001; Maher and Dolnick Nuc. Acid. Res. 16:3341-3358, 1988). However, seemingly small changes in oligonucleotide sequence, chemistry and motif can make large differences in one or more of the many properties required for clinical development (Seth et al. J. Med. Chem. 2009, 52, 10; Egli et al. J. Am. Chem. Soc. 2011, 133, 16642).

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics.

In certain embodiments, a double-stranded compound comprises a first strand comprising the nucleobase sequence complementary to a target region of a GYS1 nucleic acid and a second strand. In certain embodiments, the double-stranded compound comprises ribonucleotides in which the first strand has uracil (U) in place of thymine (T) and is complementary to a target region. In certain embodiments, a double-stranded compound comprises (i) a first strand comprising a nucleobase sequence complementary to a target region of a GYS1 nucleic acid, and (ii) a second strand. In certain embodiments, the double-stranded compound comprises one or more modified nucleotides in which the 2′ position in the sugar contains a halogen (such as fluorine group; 2′-F) or contains an alkoxy group (such as a methoxy group; 2′-OMe). In certain embodiments, the double-stranded compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the double-stranded compound comprises one or more linkages between adjacent nucleotides other than a naturally-occurring phosphodiester linkage. Examples of such linkages include phosphoramide, phosphorothioate, and phosphorodithioate linkages. The double-stranded compounds may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000. In certain embodiments, the first strand of the double-stranded compound is an siRNA guide strand and the second strand of the double-stranded compound is an siRNA passenger strand. In certain embodiments, the second strand of the double-stranded compound is complementary to the first strand. In certain embodiments, each strand of the double-stranded compound consists of 16, 17, 18, 19, 20, 21, 22, or 23 linked nucleosides.

In certain embodiments, a single-stranded compound described herein can comprise any of the oligonucleotide sequences targeted to GYS1 described herein. In certain embodiments, such a single-stranded compound is a single-stranded RNAi (ssRNAi) compound. In certain embodiments, a ssRNAi compound comprises the nucleobase sequence complementary to a target region of a GYS1 nucleic acid. In certain embodiments, the ssRNAi compound comprises ribonucleotides in which uracil (U) is in place of thymine (T). In certain embodiments, ssRNAi compound comprises a nucleobase sequence complementary to a target region of a GYS1 nucleic acid. In certain embodiments, a ssRNAi compound comprises one or more modified nucleotides in which the 2′ position in the sugar contains a halogen (such as fluorine group; 2′-F) or contains an alkoxy group (such as a methoxy group; 2′-OMe). In certain embodiments, a ssRNAi compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the ssRNAi compound. In certain embodiments, the ssRNAi compound comprises one or more linkages between adjacent nucleotides other than a naturally-occurring phosphodiester linkage. Examples of such linkages include phosphoramide, phosphorothioate, and phosphorodithioate linkages. The ssRNAi compounds may also be chemically modified nucleic acid molecules as taught in U.S. Pat. No. 6,673,661. In other embodiments, the ssRNAi contains a capped strand, as disclosed, for example, by WO 00/63364, filed Apr. 19, 2000. In certain embodiments, the ssRNAi compound consists of 16, 17, 18, 19, 20, 21, 22, or 23 linked nucleosides.

In certain embodiments, compounds described herein comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

### Certain Mechanisms

In certain embodiments, compounds described herein comprise or consist of modified oligonucleotides. In certain embodiments, compounds described herein are antisense compounds. In certain embodiments, such antisense compounds comprise oligomeric compounds. In certain embodiments, compounds described herein are capable of hybridizing to a target nucleic acid, resulting in at least one antisense activity. In certain embodiments, compounds described herein selectively affect one or more target nucleic acid. Such selective compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in the gap of a gapmer is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain such embodiments, the target nucleic acid is selected from: an mRNA and a pre-mRNA, including intronic, exonic and untranslated regions. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

Human gene sequences that encode GYS1 include, without limitation, the following gene sequences: RefSeqNo. NM_002103.4 (SEQ ID NO: 2), RefSeqNo. NM_001161587.1 (SEQ ID NO: 3), RefSeqNo. NR_027763.1 (SEQ ID NO: 4), RefSeqNo. AK303712.1 (SEQ ID NO: 5), and the complement of RefSeqNo. NC_000019.10 truncated from nucleotides 48965001 to 48996000 (SEQ ID NO: 6).

### Hybridization

In some embodiments, hybridization occurs between a compound disclosed herein and a GYS1 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the compounds provided herein are specifically hybridizable with a GYS1 nucleic acid.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, compounds described herein comprise or consist of modified oligonucleotides. In certain embodiments, compounds described herein are antisense compounds. In certain embodiments, compounds comprise oligomeric compounds. Non-complementary nucleobases between a compound and a GYS1 nucleic acid may be tolerated provided that the compound remains able to specifically hybridize to a target nucleic acid. Moreover, a compound may hybridize over one or more segments of a GYS1 nucleic acid such that intervening or adjacent segments are not involved in the hybridization event (e.g., a loop structure, mismatch or hairpin structure).

In certain embodiments, the compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a GYS1 nucleic acid, a target region, target segment, or specified portion thereof. Percent complementarity of a compound with a target nucleic acid can be determined using routine methods.

For example, a compound in which 18 of 20 nucleobases of the compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a compound which is 18 nucleobases in length having four non-complementary nucleobases which are flanked by two regions of complete complementarity with the target nucleic acid would have 77.8% overall complementarity with the target nucleic acid and would thus fall within the scope of the present invention. Percent complementarity of a compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, compounds described herein, or specified portions thereof, are fully complementary (i.e. 100% complementary) to a target nucleic acid, or specified portion thereof. For example, a compound may be fully complementary to a GYS1 nucleic acid, or a target region, or a target segment or target sequence thereof. As used herein, “fully complementary” means each nucleobase of a compound is capable of precise base pairing with the corresponding nucleobases of a target nucleic acid. For example, a 20 nucleobase compound is fully complementary to a target sequence that is 400 nucleobases long, so long as there is a corresponding 20 nucleobase portion of the target nucleic acid that is fully complementary to the compound. Fully complementary can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

The location of a non-complementary nucleobase may be at the 5′ end or 3′ end of the compound. Alternatively, the non-complementary nucleobase or nucleobases may be at an internal position of the compound. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, compounds described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a GYS1 nucleic acid, or specified portion thereof.

In certain embodiments, compounds described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a GYS1 nucleic acid, or specified portion thereof.

In certain embodiments, compounds described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of a compound. In certain embodiments, the compounds are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment. In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 11, 12, 13, 14, 15, 16, 17, 18, 19, 20, or more nucleobase portion of a target segment, or a range defined by any two of these values.

### Identity

The compounds provided herein may also have a defined percent identity to a particular nucleotide sequence, SEQ ID NO, or compound represented by a specific Isis number, or portion thereof. In certain embodiments, compounds described herein are antisense compounds or oligomeric compounds. In certain embodiments, compounds described herein are modified oligonucleotides. As used herein, a compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of an compound is calculated according to the number of bases that have identical base pairing relative to the sequence to which it is being compared.

In certain embodiments, compounds described herein, or portions thereof, are, or are at least, 70%, 75%, 80%, 85%, 90%, 91%, 92%, 93%, 94%, 95%, 96%, 97%, 98%, 99% or 100% identical to one or more of the compounds or SEQ ID NOs, or a portion thereof, disclosed herein. In certain embodiments, compounds described herein are about 70%, 75%, 80%, 85%, 90%, 91%, 92%, 93%, 94%, 95%, 96%, 97%, 98%, or 99% identical, or any percentage between such values, to a particular nucleotide sequence, SEQ ID NO, or compound represented by a specific Isis number, or portion thereof, in which the compounds comprise an oligonucleotide having one or more mismatched nucleobases. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 2, 3, 4, 5, 6, 7, 8, 9, 10, 11, or 12 from the 3′-end of the oligonucleotide.

In certain embodiments, compounds described herein are antisense compounds. In certain embodiments, a portion of the compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, compounds described herein are oligonucleotides. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Certain Modified Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

### A. Modified Nucleosides

Modified nucleosides comprise a modified sugar moiety or a modified nucleobase or both a modified sugar moiety and a modified nucleobase.

### 1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for linearly non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or 2′-non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a linear 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a linear 2′-substituent group selected from: F, OCH₃, and OCH₂CH₂OCH₃.

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 91999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

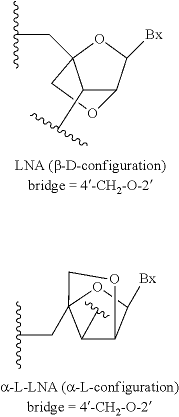

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see e.g., Leumann, C J. Bioorg. & Med. Chem. 2002, 10, 841-854), fluoro HNA:

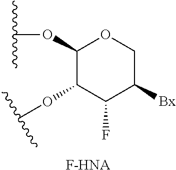

(“F-HNA”, see e.g., Swayze et al., U.S. Pat. No. 8,088,904; Swayze et al., U.S. Pat. No. 8,440,803; Swayze et al., U.S.; and Swayze et al., U.S. Pat. No. 9,005,906, F-HNA can also be referred to as a F-THP or 3′-fluoro tetrahydropyran), and nucleosides comprising additional modified THP compounds having the formula:

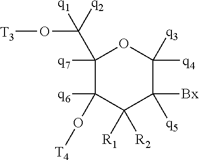

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

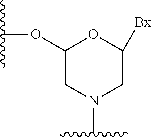

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### 2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to compounds described herein.

In certain embodiments, compounds described herein comprise modified oligonucleotides. In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimi¬dines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., Pat. No. 6,166,199; and Matteucci et al., U.S. Pat. No. 6,005,096.

In certain embodiments, compounds targeted to a GYS1 nucleic acid comprise one or more modified nucleobases. In certain embodiments, the modified nucleobase is 5-methylcytosine. In certain embodiments, each cytosine is a 5-methylcytosine.

### Modified Internucleoside Linkages

The naturally occurring internucleoside linkage of RNA and DNA is a 3′ to 5′ phosphodiester linkage. In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases.

In certain embodiments, compounds targeted to a GYS1 nucleic acid comprise one or more modified internucleoside linkages. In certain embodiments, the modified internucleoside linkages are phosphorothioate linkages. In certain embodiments, each internucleoside linkage of the compound is a phosphorothioate internucleoside linkage.

In certain embodiments, compounds described herein comprise oligonucleotides. Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH2-N(CH3)-O—CH2-), thiodiester, thionocarbamate (—O—C(═O)(NH)—S—); siloxane (—O—SiH2-O—); and N,N′-dimethylhydrazine (—CH2-N(CH3)-N(CH3)-). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH2-N(CH3)-O-5′), amide-3 (3′-CH2-C(═O)—N(H)-5′), amide-4 (3′-CH2-N(H)—C(═O)-5′), formacetal (3′-O—CH2-O-5′), methoxypropyl, and thioformacetal (3′-S—CH2-O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosponate linkages. In certain embodiments, oligonucleotides having a gapmer nucleoside motif comprise a linkage motif comprising all phosphorothioate linkages except for one or two methylphosponate linkages. In certain embodiments, one methylphosponate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

### B. Certain Motifs

In certain embodiments, compounds described herein comprise oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

### 2. Certain Nucleobase Motifs

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### 3. Certain Internucleoside Linkage Motifs

In certain embodiments, compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

### C. Certain Modified Oligonucleotides

In certain embodiments, compounds described herein comprise modified oligonucleotides. In certain embodiments, the above modifications (sugar, nucleobase, internucleoside linkage) are incorporated into a modified oligonucleotide. In certain embodiments, modified oligonucleotides are characterized by their modification, motifs, and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

### Compositions and Methods for Formulating Pharmaceutical Compositions

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In certain embodiments, the present invention provides pharmaceutical compositions comprising one or more compounds or a salt thereof. In certain embodiments, the compounds are antisense compounds or oligomeric compounds. In certain embodiments, the compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more compound. In certain embodiments, such pharmaceutical composition consists of a sterile saline solution and one or more compound. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises one or more compound and phosphate-buffered saline (PBS). In certain embodiments, a pharmaceutical composition consists of one or more compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

A compound described herein targeted to a GYS1 nucleic acid can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to a GYS1 nucleic acid and a pharmaceutically acceptable diluent. In certain embodiments, the pharmaceutically acceptable diluent is water. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a compound which are cleaved by endogenous nucleases within the body, to form the active compound.

In certain embodiments, the compounds or compositions further comprise a pharmaceutically acceptable carrier or diluent.

### ADVANTAGES OF CERTAIN EMBODIMENTS

Provided herein, for the first time, are methods and compositions for the modulation of a GYS1 nucleic acid that can treat, delay, prevent and/or ameliorate Lafora disease, or a physiological marker thereof. In a particular embodiment, for the first time, GYS1 inhibitors (e.g., oligonucleotides targeting a nucleic acid encoding GYS1) are provided for decreasing seizures, decreasing myoclonus or muscle spasms, alleviating difficulty in walking, reducing, preventing the onset of or treating dementia, alleviating difficulties in speech, reducing or preventing the onset of visual hallucinations, treating, reducing or preventing the onset of progressive neurologic degeneration, reducing ataxia, or a combination thereof in an animal.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Antisense Inhibition of Mouse GYS1 in B16-F10 Cells

Three hundred antisense oligonucleotides were screened in B16-F10 cells. The studies described below are a representative of these extensive experiments.

Antisense oligonucleotides were designed targeting a GYS1 nucleic acid and were tested for their effects on GYS1 mRNA in vitro. Cultured B16-F10 cells were transfected using electroporation with 7,000 nM antisense oligonucleotide. After a treatment period of approximately 24 hours, RNA was isolated from the cells and GYS1 mRNA levels were measured by quantitative real-time PCR. Mouse primer probe set RTS4382 (forward sequence TGATGAAGAGAGCCATCTTTGC, designated herein as SEQ ID NO: 7; reverse sequence AGGAGTCGTCCAGCATGTTGT, designated herein as SEQ ID NO: 8; probe sequence ACTCAGCGGCAGTCTTTCCCACCA, designated herein as SEQ ID NO: 9) was used to measure mRNA levels. GYS1 mRNA levels were adjusted according to total RNA content, as measured by RIBOGREEN®. Results are presented as percent inhibition of GYS1, relative to untreated control cells.

The chimeric antisense oligonucleotides in the Table below were designed as 5-10-5 MOE gapmers. The gapmers are 20 nucleosides in length, wherein the central gap segment comprises of ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate (denoted herein as ‘s’) and phosphate ester linkages (denoted herein as ‘o’). The linkage chemistry is denoted at ‘soooossssssssssooss’. All cytosine residues throughout each gapmer are 5-methylcytosines.

“Start site” indicates the 5′-most nucleoside to which the gapmer is targeted in the mouse genomic sequence. “Stop site” indicates the 3′-most nucleoside to which the gapmer is targeted mouse genomic sequence. Each gapmer listed in the Tables below is targeted to the mouse GYS1 genomic sequence, designated herein as SEQ ID NO: 1 (RefSeq No. NC_000073.6 truncated from nucleotides 45432001 to 45460000).

TABLE 1
Inhibition of mouse GYS1 mRNA by 5-10-5 MOE
gapmers targeting SEQ ID NO: 1

| | SEQ | SEQ | | | |
|---|---|---|---|---|---|
| | ID | ID | | | |
| | NO: 1 | NO: 1 | | % | SEQ |
| ISIS | Start | Stop | | inhibi- | ID |
| NO | Site | Site | Sequence | tion | NO |
| 648122 | 2870 | 2889 | CCGACTCAGGTAGGGTGAGC | 58 | 10 |
| 648127 | 2963 | 2982 | CTTGGTGACCGGTAGAGTTA | 21 | 11 |
| 648130 | 3042 | 3061 | GAGAGGCATGGCTACTGCGG | 68 | 12 |
| 648131 | 3047 | 3066 | CGGCTGAGAGGCATGGCTAC | 45 | 13 |
| 648135 | 3083 | 3102 | TCTTCCAATCCTGGAAGCGA | 8 | 14 |
| 648154 | 4596 | 4615 | ATGGTCCCACCAGATAGTAG | 78 | 15 |
| 648155 | 4601 | 4620 | CGTGTATGGTCCCACCAGAT | 73 | 16 |
| 648194 | 8013 | 8032 | AGGTGTTGAGCCTCGATTGC | 45 | 17 |
| 648222 | 12632 | 12651 | TGACTGTATTGGCTGTGTCC | 57 | 18 |
| 648223 | 12637 | 12656 | CTCCTTGACTGTATTGGCTG | 57 | 19 |
| 648226 | 12658 | 12677 | GTAGAGCTTCCTCCCAAATT | 39 | 20 |
| 648258 | 19754 | 19773 | CCTCCGATCCAGAATGTAAA | 24 | 21 |
| 648267 | 19884 | 19903 | ACTTCCAATCTAGCAAGTCC | 28 | 22 |
| 648291 | 23125 | 23144 | TCCCGTGGCTCTTCCTCATC | 11 | 23 |
| 648298 | 23240 | 23259 | TGTGGAGGAGGAACAGGAGG | 0 | 24 |
| 648299 | 23247 | 23266 | TGCCACCTGTGGAGGAGGAA | 22 | 25 |
| 648302 | 23284 | 23303 | CTGGAGGGCCCAGTGTCCAC | 45 | 26 |
| 648303 | 23289 | 23308 | GTGAGCTGGAGGGCCCAGTG | 32 | 27 |
| 648306 | 23406 | 23425 | CATAGGCCCTCTGCGAGAGG | 52 | 28 |
| 648307 | 23411 | 23430 | ATCTGCATAGGCCCTCTGCG | 52 | 29 |
| 648310 | 23426 | 23445 | TTCAGGCACCCTCCCATCTG | 53 | 30 |
| 648311 | 23447 | 23466 | ACTCAAGAGTCTGGAGTGGG | 26 | 31 |
| 648314 | 23502 | 23521 | GGCTGGAGTGTCTGAAACAG | 64 | 32 |
| 648315 | 23513 | 23532 | TGGAGCTCAAGGGCTGGAGT | 23 | 33 |
| 648318 | 23555 | 23574 | CCAAGAAAGGCACGGCGGCG | 74 | 34 |
| 648319 | 23597 | 23616 | CTGGAGACTCCAGATCAGTG | 50 | 35 |
| 648322 | 23638 | 23657 | AAACAATGGCAGATGCCTGG | 40 | 36 |
| 648323 | 23659 | 23678 | CCTAAAACCTCTGGCATTGA | 42 | 37 |
| 648326 | 23683 | 23702 | CCTGGAAGCCAATAAACCAG | 64 | 38 |
| 648327 | 23690 | 23709 | GCCACAGCCTGGAAGCCAAT | 76 | 39 |
| 648330 | 23774 | 23793 | GCCACACAGAATCCAACATG | 80 | 40 |
| 648331 | 23781 | 23800 | TCGGGAAGCCACACAGAATC | 34 | 41 |
| 648334 | 23813 | 23832 | CCTGAAATGTCCTAACTCTG | 49 | 42 |
| 648335 | 23819 | 23838 | TTAATCCCTGAAATGTCCTA | 21 | 43 |
| 648338 | 23870 | 23889 | AATCTGTCGACAGAGCTACT | 72 | 44 |
| 648339 | 23877 | 23896 | GACAAGCAATCTGTCGACAG | 79 | 45 |
| 648342 | 23940 | 23959 | TGTGTATCACCGCACCAGGT | 65 | 46 |
| 648346 | 24097 | 24116 | GAAATGGAGGACCGTGAGCA | 62 | 47 |
| 648347 | 24165 | 24184 | TGCTCCTTTGAAGAACACAC | 39 | 48 |
| 648350 | 24193 | 24212 | GCAGAAAGGTGTCTGGTCCA | 75 | 49 |
| 648351 | 24196 | 24215 | AAGGCAGAAAGGTGTCTGGT | 30 | 50 |
| 648354 | 24275 | 24294 | TGACAGACATTCTGCCCTCA | 65 | 51 |
| 648355 | 24298 | 24317 | AGTGGGCTGAGCACTTGTGG | 39 | 52 |
| 648359 | 24330 | 24349 | AGCCACTGGGACCCAGAACC | 18 | 53 |
| 648362 | 24408 | 24427 | TTCAAGAAGCCGGTGGGCTC | 45 | 54 |
| 648363 | 24437 | 24456 | GCAGAAAGGCCTCGAGGTAC | 59 | 55 |
| 648371 | 3326 | 3345 | CCCCCCAGGGCCTAGGACGC | 65 | 56 |
| 648375 | 3961 | 3980 | ACAGCATTGAGTCTGCCATC | 60 | 57 |
| 648382 | 5607 | 5626 | TGGCCTGACTGGATGCTGGA | 7 | 58 |
| 648390 | 5778 | 5797 | ATTGATCTAACTCTGTCCCA | 32 | 59 |
| 648394 | 6413 | 6432 | ATCCTTGGATTAAAAGAGTG | 42 | 60 |
| 648395 | 6775 | 6794 | GACCAAAACTCCCAGATTTC | 51 | 61 |
| 648399 | 8063 | 8082 | AGCCACATGTAGGGACCACA | 49 | 62 |
| 648402 | 10168 | 10187 | CATGCTTCATTTCTTTATTG | 81 | 63 |
| 648403 | 10380 | 10399 | GGCCCATGCTTCATTTCTTT | 63 | 64 |
| 648406 | 11693 | 11712 | TCAGAGATAGCCAGAGAGAG | 51 | 65 |
| 648407 | 12197 | 12216 | CCCTACTGTCTCATGACTTA | 39 | 66 |
| 648414 | 12895 | 12914 | GAGGCCTCAGCAAATGCCAG | 29 | 67 |
| 648415 | 13163 | 13182 | CCTCCAGCAATGTATTTTAA | 35 | 68 |
| 648418 | 14487 | 14506 | AGGAATCAGAGGGTTCTGTG | 50 | 69 |
| 648419 | 14886 | 14905 | AGCCCTCTCTTTTATGACAA | 13 | 70 |
| 648422 | 16141 | 16160 | ACAAGCTAAAGACTTAAACT | 29 | 71 |
| 648423 | 17471 | 17490 | GATTTGCAAGTGACTCTCAA | 69 | 72 |
| 648426 | 20057 | 20076 | TGGATTCCCTCTGTAGATCA | 59 | 73 |
| 648427 | 20466 | 20485 | TGTCTCTAGCTCTGACAACA | 45 | 74 |
| 648430 | 22024 | 22043 | CCAGATGCTATTTCTAGATT | 88 | 75 |
| 648431 | 22395 | 22414 | ACTGCTGGAGTCCCCAGCAA | 3 | 76 |

### Example 2: Dose-Dependent Antisense Inhibition of Mouse GYS1 in B16-F10 Cells

Gapmers from Example 1 exhibiting significant in vitro inhibition of GYS1 mRNA were selected and tested at various doses in B16-F10 cells. Cells were plated at a density of 20,000 cells per well and transfected using electroporation with 0.625 μM, 1.25 μM, 5.00 μM, and 10.0 μM concentrations of antisense oligonucleotide. After a treatment period of approximately 16 hours, RNA was isolated from the cells and GYS1 mRNA levels were measured by quantitative real-time PCR. Mouse primer probe set RTS4382 was used to measure mRNA levels. GYS1 mRNA levels were adjusted according to total RNA content, as measured by RIBOGREEN®. The half maximal inhibitory concentration (IC₅₀) of each oligonucleotide is presented. GYS1 mRNA levels were significantly reduced in a dose-dependent manner in antisense oligonucleotide treated cells.

Two antisense oligonucleotides, ISIS 648327 and ISIS 648402 were selected for further experimentation in vivo.

TABLE 2
Dose dependent inhibition by antisense oligonucleotides targeting GYS1

| ISIS No | 625.0 nM | 1250.0 nM | 2500.0 nM | 5000.0 nM | 10000.0 nM | IC₅₀ (μM) |
|---|---|---|---|---|---|---|
| 648339 | 19 | 38 | 58 | 77 | 93 | 1.9 |
| 648327 | 32 | 44 | 66 | 82 | 90 | 1.4 |
| 648155 | 18 | 42 | 62 | 76 | 90 | 1.9 |
| 648423 | 12 | 26 | 38 | 64 | 70 | 3.6 |
| 648371 | 38 | 55 | 78 | 82 | 93 | 0.9 |
| 648430 | 4 | 9 | 25 | 31 | 53 | 10.7 |
| 648402 | 53 | 42 | 62 | 84 | 89 | 0.9 |
| 648330 | 35 | 42 | 62 | 74 | 89 | 1.5 |
| 648154 | 21 | 32 | 55 | 69 | 83 | 2.3 |
| 648350 | 21 | 29 | 58 | 70 | 85 | 2.3 |

### Example 3: Intracerebroventricular Administration of Antisense Oligonucleotides Against GYS1 mRNA

C57BL/6 mice were treated with ISIS oligonucleotides via intracerebroventricular (ICV) administration to a defined mouse brain area, the right lateral ventricle, for the purpose of evaluating the efficacy of ICV dosing in mice.

### Treatment

Groups of four C57BL/6 mice each were administered ISIS 648154, ISIS 648155, ISIS 648327, ISIS 648330, ISIS 648339, ISIS 648350, ISIS 648371, or ISIS 648402 at 300 μg delivered as an ICV bolus injection. A control group of 4 mice were similarly treated with PBS. The animals were euthanized after 2 weeks.

### RNA Analysis

RNA was extracted from the right hemisphere of the cortex, hippocampus, and the cerebellar sections for real-time PCR analysis of GYS-1 mRNA levels. Murine GYS-1 mRNA levels were measured using the primer probe set RTS4382. Results were calculated as percent inhibition of murine GYS-1 mRNA expression compared to the control and are presented in the Table below. Of the antisense oligonucleotides tested, ISIS 648327 and ISIS 648402 were utilized in further studies.

TABLE 3
Percent inhibition by antisense oligonucleotides targeting GYS1

| ISIS No | Cortex | Hippocampus | Cerebellum |
|---|---|---|---|
| 648154 | 0 | 7 | 24 |
| 648155 | 6 | 0 | 30 |
| 648327 | 60 | 40 | 27 |
| 648330 | 21 | 21 | 33 |
| 648339 | 16 | 0 | 2 |
| 648350 | 24 | 12 | 10 |
| 648371 | 3 | 0 | 21 |
| 648402 | 51 | 21 | 45 |

### Example 4: Effect of Antisense Inhibition of GYS1 in Mice Models for Lafora Disease

Mutations in the EPM2A gene, encoding a dual-specificity phosphatase (Laforin) or in the EPM2B gene, encoding ubiquitin E3 ligase malin, cause Lafora disease (LD). The phenotype of targeted disruption of the Epm2a or Epm2b murine genes is comparable to the phenotype of human LD resulting from the same genetic defect. Both Epm2a^{-/-} and Epm2b^{-/-} mice show altered motor activity, impaired motor coordination, episodic memory deficit, and myoclonus (Garcia-Cabrero A. M. et al., 2012. J. Neuropathol. Exp. Neurol. 71: 413-421). Neurologic alterations observed in the mutants were comparable and correlated with the accumulation of abundant Lafora bodies in the cerebral cortex, the hippocampus, the basal ganglia, the cerebellum, and the brainstem, suggesting that these inclusions could cause cognitive and behavioral deterioration. Thus, both Epm2a^{-/-} and Epm2b^{-/-} mice exhibit many pathologic aspects seen in patients with Lafora disease and serve as mouse models for the disease.

### Epm2a^{-/-} Mice

### Treatment

The effects of antisense inhibition of GYS1 were investigated in Epm2a^{-/-} mice. The mice have been previously described (Pedersen, B. A. et al., Ann. Neurol. 74: 297-300, 2013; Turnball, J. et al., PLoS Genet. 7: e1002037 (2011). The mice were randomly divided into 4 groups of 6 mice each. Two groups of mice were injected intracerebroventricularly with 300 μg of ISIS 648327 or ISIS 648402 at 1 month and 2 months of age. A third group of mice were injected intracerebroventricularly with 300 μg of ISIS 676630 (CCTATAGGACTATCCAGGAA, 5-10-5 MOE gapmer with phosphorothioate and phosphate internucleoside linkages and with no known murine target; SEQ ID NO: 77) at 1 month and 2 months of age. A control group of mice was injected intracerebroventricularly with PBS at 1 month and 2 months of age. The mice were sacrificed at 3 months by cervical dislocation, one cerebral hemisphere was snap-frozen in liquid nitrogen for biochemical analysis; the other was immersed in formalin for histopathology.

### RNA Analysis

RNA was extracted from cerebral hemisphere of the mice for RT-PCR analysis of murine GYS1 expression. The data was normalized to GAPDH. The results are presented in the Table below and demonstrate the in vivo inhibition of GYS1 by antisense oligonucleotide.

TABLE 4
In vivo inhibition (% of PBS control)
of mouse GYS1 mRNA levels

| | % |
|---|---|
| Control oligo | 23 |
| ISIS 648327 | 76 |
| ISIS 648402 | 84 |

### Protein Analysis

Western blot analysis of murine GYS1 protein expression was quantitated. The results are presented relative to the housekeeping gene, GAPDH, in the Table below and demonstrate the in vivo inhibition of GYS1 protein levels by antisense oligonucleotide.

TABLE 5
Mouse GYS1 protein levels (% relative to GAPDH)

| | % |
|---|---|
| PBS | 14 |
| Control oligo | 11 |
| ISIS 648327 | 2 |
| ISIS 648402 | 1 |

### Glycogen Level Analysis

Lafora bodies were quantified by biochemical measurement of total brain glycogen, as previously described (Turball, J. et al. PLoS Genet. 7: e1002037, 2011). This mice model typically has two-fold increased brain glycogen at 3 months of age. The results are presented in the Table below and demonstrate the effect of inhibition of GYS1 on brain glycogen levels. The data indicates that brain glycogen levels were normalized by both antisense oligonucleotides. Glycogen levels of wild-type mice are also shown for comparison. The PBS and control oligonucleotide groups have high glycogen content, representing the levels in Lafora disease.

TABLE 6
Brain glycogen levels (μmol/g tissue)

| | |
|---|---|
| PBS | 8.7 |
| Control oligo | 7.8 |
| ISIS 648327 | 3.4 |
| ISIS 648402 | 2.8 |
| Wild-type | 2.4 |

### Lafora Bodies Analysis

Lafora bodies (LB) were visualized by staining diastase-pretreated brain sections with periodic acid-Schiff. In animals treated with ISIS 648327, LB were completely eliminated from all brain regions. In animals treated with ISIS 648402, LB were almost completely eliminated from all brain regions. The data thus demonstrates that antisense inhibition of GYS1 significantly reduced or completely eliminated LB from the brain.

### Epm2b^{-/-} Mice

### Treatment

The effects of antisense inhibition of GYS1 were investigated in Epm2b^{-/-} mice. The mice have been previously described (Pedersen, B. A. et al., Ann. Neurol. 74: 297-300, 2013; Turnball, J. et al., PLoS Genet. 7: e1002037 (2011). The mice were randomly divided into 3 groups of 6 mice each. One group of mice was injected intracerebroventricularly with 300 μg of ISIS 648327 at 1 month and 2 months of age. A second group of mice were injected intracerebroventricularly with 300 μg of ISIS 676630 at 1 month and 2 months of age. A control group of mice was injected intracerebroventricularly with PBS at 1 month and 2 months of age. The mice were sacrificed at 3 months by cervical dislocation, one cerebral hemisphere was span-frozen in liquid nitrogen for biochemical analysis; the other was immersed in formalin for histopathology.

### RNA Analysis

RNA was extracted from cerebral hemisphere of the mice for RT-PCR analysis of murine GYS1 expression. The data was normalized to GAPDH. The results are presented in the Table below and demonstrate the in vivo inhibition of GYS1 by antisense oligonucleotide.

TABLE 7
In vivo inhibition (% of PBS control)
of mouse GYS1 mRNA levels

| | % |
|---|---|
| Control oligo | 17 |
| ISIS 648327 | 56 |

### Protein Analysis

Western blot analysis of murine GYS1 protein expression was quantitated. The results are presented relative to the housekeeping gene, GAPDH, in the Table below and demonstrate the in vivo inhibition of GYS1 protein levels by antisense oligonucleotide.

TABLE 8
Mouse GYS1 protein levels (% relative to GAPDH)

| | % |
|---|---|
| PBS | 42 |
| Control oligo | 34 |
| ISIS 648327 | 6 |

### Glycogen Level Analysis

Lafora bodies were quantified by biochemical measurement of total brain glycogen, as previously described (Turball, J. et al. PLoS Genet. 7: e1002037, 2011). The results are presented in the Table below and demonstrate the effect of inhibition of GYS1 on brain glycogen levels. The data indicates that brain glycogen levels were normalized by both antisense oligonucleotides. Glycogen levels of wild-type mice are also shown for comparison. As previously described in DePaoli-Roach et al (J. Biochem. 13: 25372-25381, 2010), the abnormal glycogen accumulation as not as high in in Epm2b^{-/-} mice as in in Epm2a^{-/-} mice.

### Example 5: Effect of Antisense Inhibition of GYS1 in Mice Models for Adult Polyglucosan Body Disease

The effects of antisense inhibition of GYS1 were investigated in APBD mice or Gbe1 Y329S mice. The mice have been previously described (Akman, O. H. et al., Hum. Mo. Genet. 24: 6801-6810, 2015). The mice were randomly divided into 4 groups. Two groups of mice were injected intracerebroventricularly with 300 μg of ISIS 648402 or ISIS 648327 at 1 month and 2 months of age. A third group of mice were injected intracerebroventricularly with 300 μg of control oligonucleotide ISIS 676630 at 1 month and 2 months of age. Another group of mice was injected intracerebroventricularly with PBS at 1 month and 2 months of age. The mice were sacrificed at 3 months by cervical dislocation, one cerebral hemisphere was span-frozen in liquid nitrogen for biochemical analysis; the other was immersed in formalin for histopathology.

### RNA Analysis

RNA was extracted from cerebral hemisphere of the mice for RT-PCR analysis of murine GYS1 expression. The data was normalized to GAPDH. The results are presented in the Table below and demonstrate the in vivo inhibition of GYS1 by antisense oligonucleotide.

TABLE 9
In vivo inhibition (% of PBS control)
of mouse GYS1 mRNA levels

| | % |
|---|---|
| Control oligo | 9 |
| ISIS 648402 | 59 |
| ISIS 648327 | 69 |

### Protein Analysis

Western blot analysis of murine GYS1 protein expression was quantitated. The results are presented relative to the housekeeping gene, GAPDH, in the Table below and demonstrate the in vivo inhibition of GYS1 protein levels by antisense oligonucleotide.

TABLE 10
Mouse GYS1 protein levels (% relative to GAPDH)

| | % |
|---|---|
| PBS | 10.0 |
| Control oligo | 16.0 |
| ISIS 648402 | 0.8 |
| ISIS 648327 | 3.1 |

### Glycogen Level Analysis

Lafora bodies were quantified by biochemical measurement of total brain glycogen, as previously described (Turball, J. et al. PLoS Genet. 7: e1002037, 2011). The results are presented in the Table below and demonstrate the effect of inhibition of GYS1 on brain glycogen levels.

TABLE 11
Brain glycogen levels (μmol/g tissue)

| | |
|---|---|
| PBS | 1.5 |
| Control oligo | 1.6 |
| ISIS 648402 | 1.1 |
| ISIS 648327 | 0.8 |

### Example 6: Effect of Antisense Inhibition of GYS1 in the Treatment of a Glycogen Storage Disease

The effects of antisense inhibition of GYS1 were investigated in aged EMP2A (Laforin) knockout mice. Lafora bodies begin to appear in the brain by two months, behavioral abnormalities are detected at 4 months, and by 9 months, the animals have myoclonic seizures, ataxia, and EEG activity (Wang W. et al., Arch. Biochem. Biophys. 457: 264, 2007).

Eight months old Epm2a^{-/-} mice were randomly divided into 3 groups. Two groups of mice were injected intracerebroventricularly with 300 μg of ISIS 648402 or the control oligonucleotide at 8 months, 9.5 months, 11 months, and 12.5 months of age. Another group of mice was injected intracerebroventricularly with PBS at 8 months, 9.5 months, 11 months, and 12.5 months of age. The mice were sacrificed at 14 months by cervical dislocation, one cerebral hemisphere was span-frozen in liquid nitrogen for biochemical analysis; the other was immersed in formalin for histopathology.

### RNA Analysis

RNA was extracted from cerebral hemisphere of the mice for RT-PCR analysis of murine GYS1 expression. The data was normalized to GAPDH. The results are presented in the Table below and demonstrate the in vivo inhibition of GYS1 by antisense oligonucleotide.

TABLE 12
In vivo inhibition (% of PBS control)
of mouse GYS1 mRNA levels

| | % |
|---|---|
| Control oligo | 0 |
| ISIS 648327 | 54 |

### Protein Analysis

Western blot analysis of murine GYS1 protein expression was quantitated. The results are presented relative to the housekeeping gene, GAPDH, in the Table below and demonstrate the in vivo inhibition of GYS1 protein levels by antisense oligonucleotide.

TABLE 13
Mouse GYS1 protein levels (relative to GAPDH)

| | % |
|---|---|
| Untreated mice at 8 months | 22.1 |
| Control oligo-treated mice at 14 months | 13.2 |
| ISIS 648327-treated mice at 14 months | 0.50 |

### Glycogen Level Analysis

Total brain glycogen levels and Lafora bodies quantification in the hippocampus were measured. Brain tissue of mice at 8 months shows glycogen levels and accumulation of Lafora bodies in the hippocampus, implying a robust phenotype of glycogen storage disease in these mice at that age. The results are presented in the Tables below and demonstrate the effect of inhibition of GYS1 and the significant reduction in Lafora bodies in the hippocampus.

TABLE 14
Lafora bodies in hippocampus (%)

| | |
|---|---|
| Untreated mice at 8 months | 7.02 |
| Control oligo-treated mice at 14 months | 8.04 |
| ISIS 648327-treated mice at 14 months | 2.67 |

TABLE 15
Brain glycogen levels (μmol/g tissue)

| | |
|---|---|
| Untreated mice at 8 months | 5.9 |
| Control oligo-treated mice at 14 months | 7.5 |
| ISIS 648327-treated mice at 14 months | 3.5 |

1-64. (canceled)

65. A method of treating adult polyglucosan body disease in an individual having adult polyglucosan body disease comprising administering to the individual a compound comprising a modified oligonucleotide consisting of 15 to 30 nucleosides, wherein the modified oligonucleotide has a nucleobase sequence at least 95% complementary to an equal length portion of the nucleobase sequence of any one of SEQ ID NOs: 2-6, and wherein at least one internucleoside linkage of the modified oligonucleotide is a phosphorothioate internucleoside linkage, thereby treating the adult polyglucosan body disease in the individual.

66. The method of claim 65, wherein the compound is single-stranded.

67. The method of claim 65, wherein the modified oligonucleotide comprises at least one modified sugar moiety or at least one modified nucleobase.

68. The method of claim 67, wherein the at least one modified nucleobase is a 5-methylcytosine.

69. The method of claim 67, wherein the at least one modified sugar moiety is a bicyclic sugar moiety.

70. The method of claim 69, wherein the bicyclic sugar moiety comprises a 4′-CH(CH₃)—O-2′ bridge or a 4′-(CH₂)n-O-2′ bridge, wherein n is 1 or 2.

71. The method of claim 67, wherein the at least one modified sugar moiety is a non-bicyclic moiety.

72. The method of claim 71, wherein the non-bicyclic moiety is selected from 2′-F, 2′-OMe, and 2′-MOE.

73. The method of claim 67, wherein the at least one modified sugar moiety is a sugar surrogate.

74. The method of claim 73, wherein the sugar surrogate is selected from morpholino and PNA.

75. The method of claim 65, wherein the modified oligonucleotide is a gapmer.

76. The method of claim 65, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned immediately adjacent to and between the 5′ wing segment and the 3′ wing segment and wherein each nucleoside of each wing segment comprises a modified sugar moiety.

77. The method of claim 76, wherein the modified sugar moiety of each wing segment is a 2′-O-methyoxyethyl, and wherein each cytosine of said modified oligonucleotide is a 5-methylcytosine.

78. The method of claim 65, wherein the modified oligonucleotide comprises:
a gap segment consisting of 7-12 linked deoxynucleosides;
a 5′ wing segment consisting of 1-5 linked nucleosides; and
a 3′ wing segment consisting of 1-5 linked nucleosides;
wherein the gap segment is positioned immediately adjacent to and between the 5′ wing segment and the 3′ wing segment and wherein each nucleoside of each wing segment comprises a modified sugar moiety.

79. The method of claim 65, wherein at least one internucleoside linkage of the modified oligonucleotide is a phosphodiester internucleoside linkage.

80. The method of claim 65, wherein the modified oligonucleotide consists of 15-25, 16-20, 18-22, or 18-20 linked nucleosides.

81. The method of claim 65, wherein Lafora bodies are reduced in a cell in the individual.

82. The method of claim 65, wherein the individual is a human.

83. A method of inhibiting expression or activity of GYS1 in a cell of an individual with adult polyglucosan body disease comprising contacting the cell with a compound comprising a modified oligonucleotide consisting of 15 to 30 nucleosides, wherein the modified oligonucleotide has a nucleobase sequence at least 95% complementary to an equal length portion of the nucleobase sequence of any one of SEQ ID NOs: 2-6, and wherein at least one internucleoside linkage of the modified oligonucleotide is a phosphorothioate internucleoside linkage, thereby inhibiting expression or activity of GYS1 in the cell.

84. The method of claim 83, wherein the cell is a neuron.


SEQUENCE LISTING (77 sequences)

SEQ ID NO: 1 - DNA, 28000 bp, Mus musculus
  source 1..28000: mol_type = genomic DNA
tggactattactcaggcaggaaaaaagagtgaagctctgaccccaacacacccaggaagaccactgagaggtgctgagcgagtgggctcccacataataaagactgcacgcaggggctggagagaagggtggaggactcttggccggtcttccttccaggggaccgaggctaaactgactgcacacatgtggtggctcacagctgactctaactctggttccggagatccaataccttcttctggcctcctcaggcatagacgtatccggagggaaaacacccatacacagaaaacatggtgaataaaccctcacgttgaaggattcttttttgttgttgttgctttggtttttttcgagacagggtttctcagtgtaggtagccctggctggccttgaacttagaaatccacctgcctctgcctcccaagtgctgggattaaaggcatgcgccaccactgcccagcttgggttgaagggttcttatatgaagtatccaaaagagatgccagaaatacaaggtagactagtggttgccaaaggctcggggagggaggtgtggggacactggagggtgcctgctaggatgcacttctgcagccactggggaggctgagtcagaaagagtgcctcgagtctggagctagcccaagctaaagggtgaaaacaagagaccgataaatccaggcggtggacccttgaacatcttttcctattttttacgaacctaagaaaccacaaagctaagcggccctcaaccacagctcaggatgcctcctcctggaatggaacaaggccttccaggatagcccacagcccaggaccatttactgcctgtctcttgctgtgggagccttcaccttcctctttttgtaggagattgtgtggactgcaggagcatgagacccccaaactactgtcacacacaggcaagcaaaggcaggcccttcctcaccatggtaacagcaacagacgccactgacctccctacaacttgcctgggcccaaccccagaatcaaggacaccacaaggccacaagatttcttcactgggaagtctgaggagctagtgccaggcccaatatgcccggtctcacttcctgggctttattcgctcactgtaacattcaactaactgtgcgatggcacagacacgtgccctaggtgaggttctgaaaaatacagtctctggacaagaccccatgctctgaacccagactctatttctcttcctcactgtcacactcctggcttcggacacactttcctgtgacctgtgctctcaagcacacaacacaacttcccacttactaatcttccccagaagaccagggtaccagtctcagacctcgcaggacactgggatgagactttggaactggtcactgactacatcaagaggcagacgaagtttccagaacgcattgtgggatgaaccacgactctgtcactccaggctctggctagtcactcattcacagagaaatctgagtgtctacatgactcttccatcagacccaggattctttctgttgttgtttctctgtgtaacccttggctgtcctggaactggctctgtagaccaggctatcctcagactcacagagatccacctgcctctgcctcccaagtgcaggctttcactaccatgcctgacatctaggattctgaatacattgttgccttctctactagcttctaaagccacaatgaagctccttcctcagtggtcaatacacttcctccacatccaggagtccaggtccagccctcttctattaggcccaggcaccctccctagccctcctcctcctccaaaccaggcctcatccactgtgatcgtccctccgatctagggattcaggctctagcctgcctacctcaaacacaaaggtgcacacaccagcctgcttccctcagaccctggtatcctctcctcccaacacttcccacagatccatgtgtcctcccaagccctccttggcgacttcctcagcccccttccgtcagattcatgcattctcctctgctctcttcctttagacacatgcattctcctctgtatccattgctcagactcggtgtcctcagcatcctccctctgactcgggagtactccccagacctcctccgtcaaaccaggcatcttccttatcactcctccctcagatacaagagtcctctccagctccctccttcagactctctaagaagggtcattgtcggtcctccacatgcacccaacacttaccacggttgccatgatgcacaccaaccgccaatgcctcagcggaaacggaaatcggctaggagcgcggtctttatcctctctaggctcccggtcatccagtcatctctatggcccataagatatataaaattcctgtagggggcgtacactaggccgcgcccagcccgcgaggctaaattggaaagccggtgttctgaatcccgggtgggttcctgacgtctcttgctcctcctcctcttacctagcctcctcccccctccttcccccccgccctgcaactgctaagtccgattgcaaaattctgagcacattttgtagcaaccacacgtcctccacgatcccttccctctctgtccaaccttcctcaccaaattgggcctacagctgagattgacagctctgttcaccaatcacttccatgctgcgtagaaacaagccccgcccccgcagctccccaggagcctctggccaaggaaactggattctatccgccaataagtagacgcaatagcccccgccccccagccgccgaccaatcgtgctagaccagcagggtgggcagtgcctcctggcggccacgaggcttcactgcagctgcccgcccgattcagtgtctcagctcaccctacctgagtcggagcgctctggggcgggggtgcggtcgtgcaataggaagcggagcgccttgcaagcttcccctgggacacccgctaactctaccggtcaccaagtctgctgcgttcccagccgatctctctggtttccagttttggtgctcgaagtcccctgcccgcagtagccatgcctctcagccgcagtctctctgtgtcctcgcttccaggattggaagactgggaggatgaattcgaccccgagaacgcagtgcttttcgaggtggcctgggaggtggccaacaagggtgagaacgtcagcgttggtcccttgaccaagaagggcaggcgaggatggggccccgaaactccgtttcagacgggaaaagagttacgtggaggagtcttgggtcctgggaaaagcgcctgtgacttctgtgtccttgtgaaggggctggagtaggcgtcctaggccctggggggaagggacaagagactactcttaagatggaaggaggcttcaggttctgcaatctagggaagagggcactcagagaaagattcctgggtcttgaagggaggttttcgagggccgagagacctgagatctaaaagtggaaggagttagtgcatctaagcccaaggaacactggagcttttgggtctgtggtccagaggacgtgactgtggagttggggtgttcctaaaggttgggattcatggatttttcagggggcatagcggacctgaaccagtgtaaagccaagaggggcttggagactcaaattcttagatacaaggtgggaaagagttgggaacttgaatgtcgagtttgaaggagcccagggcctaagtcctggcttcctatgtagtgaggtgcctccctccctgtggactgggaaagctggaagtcctaagttggtcaggagctgggccctaggatgggaggaacccacccacagtatgtatgggagatcactagcttctcgctcatgagaaggctgggctgggaatcccagcagctgggccttgtggatctgaacatttccttttctggatcctggggagaaagagactaggtctctcagcatgttgggagatggcagactcaatgctgtgtactaccagggtaccagtttcaggtgagaaggctgaggacccagattcctaaatctgaggtggggtcacaggaggagcagggcccagcagtccctgtgtcccagcccagggctagcatcgttaggggccctgaaggtgtggaataataagtcaagcttccctcaggtaactgggtagctggatagggccatgtaccatttttcccactgcatctgaaagtcaagagctttagagccttcctgctcctaattctctaagaattaggagactaaagagtgcttcctgtgtcatgaccaggagtctaactggagcctgctcctccctcagcatcctgatttcagtcccctctctccataaactcaagagggctatcctcaggtttgcttttccacgacccctaccctccagtcccaacctcccctgattcccaattataggttcctgcccattttctaccccaatactacccacatacatcccattacctgccaggtctcaaagctatggaccaatggtggggttgtctgtccttttgcactttggcccacagtgggtggcatctacactgtgctgcagacgaaggcgaaggtgacaggggatgaatggggtgacaactactatctggtgggaccatacacggagcagggtgtgaggacgcaggtagagctcctggagcccccaactccggaactgaagaggactttggattccatgaacagcaagggttgtaaggtgggacacagccctacccaggggggtgggagtgggaactgccagcaggggccctagcaagaggtgaagagaaggtggaatggaggcaggaggtgtagtcagaactttcccagcaggcctagggaagaaagcgatagacgggtataggatctggtgaagtaggccagtgggtaccgggtgtcagaaaggccagatggatggagatgaatctcagcagagatcctcagacccagagactacaaacaagctggagctaggcagaaagatagtcaaagctatgtgttctgtgcacacctgcaatcccaggactacggaggcctaggcaggaggatctcggtgtgttctaagccagtctggaccacaagaaggctctgtctcacagtaaataaaataaagatgagtagccaggcagacaaaaagatggaatagagatgaagagaggagaaccaggggaaaagagtctgcggtgagcagatccatgaactaacgaggaaggggcgtggctcagaggcagagcccctgcctagaatcccccaatgaggggctaggggtgtggcccagtggcagagtccttgcctagaatctccctgtaggggtctggggttatagctcagcggtagaacccctgcctagcatggcccgtgggtttcatcctaagaaccacaaacagaagcctgttggtcccaacactacagagtaagttccaggacagccaggactaggcagagaagccctgtccgtctccccctccccccacccccacccccgccaaagaaatacaaggagaaagccagaagagagggaaggtgagatggaagaaccctagaagagatgcagatggaaagacaagtgcagaggctccacctagtcacagcctgtgttcggggttgggttccagcatccagtcaggccagaccatgtccctatgagaccccagagtggccacatccctcccccaggacagctgttcctaataatagacccccccctcccagcaaaagggcagacaacagctgggagggggaggggtgctccagtgaggccctggccagacgaagccaggttgggacagagttagatcaatggcctggagccatacctgtggtctccggaggagaatggctaatggggactgaaatagaacatcgagttttaggaaggtggcaattggtaggaaagtacagtcctggctgatagacacgtgacacaggtgacaaaacaggccaatggggagaggagaaaggtagtgggaggggttacagagggccacagatgaggccgtgggtgaggggaacgtgagacagctgagctcagttaccaggctggggatgtaagccggcgaggaatgcgggccaggtcttctgagcctccctttgcctctccctcaggtgtattttgggcgttggctgatcgaggggggacccctagtggtgctcctggatgtaggagcctcagcttgggccctggagcgctggaagggtgagctttgggacacctgcaacatcggggtaccctggtacgaccgcgaggccaatgacgctgtcctgttcggcttcctcaccacctggttcctgggtgaggtaggcggtactacagaccccacctcagccttcctacccagacttgaaaccatcatcatcatcatcaccgcttgggccagagagtgggcttcccctgggaattctgggaattgtccatccactcttttaatccaaggatcgtctctgaaaagagtctagctgcttttctcagagaccagaagtgtcaacctcagtttcatttcctctatatccaggtgtaaacacactgcgacattgtttccagggagtcctgccagaagagggttgggtcccaggaattctgggagttgctgtctactttaatatccattaccgttaagcactcagggtaacttttgatacttaccaaccttgagttggtttcagaaccagaaatctgggctttccccaccccaaacccattctagaatttttaagagaagtttttaggagacttaattgggatgccttaggacaaaaaagagctacacgtgcgttcaaggaaatctgggagttttggtcctgttttgtgcccataaaagttctactaccgctgattgaaatgctgagtcttagccgggtggtggtggcatgtacctttaatctcgggaggtagagacaagcagatccctgagttcagggcaagcctgttctacaaagtgatttccaggacagccagtgctattacacagagaaaatctgtcttgaaaaaccaaaagaagccgggcagtagtggcccacgcctttaatcccagcactccagaggcagaggcaggcagatttctgagttcgaggccagcctggtctacagagtgagttccaggacagccagggctatacagagaaaccctgtctcaaaaaaccaaaaaaaaaaacaaaaaaaaaaaccaaacaaacaaacaaaaaaagaccaagtccctgcagtagtgaccagggcttggggtgttgatccttccagttccgcgttgccagaattcataccttgccaaaaaaaaaaaaaaaaaaaaaaaaatctcattaacctctggggacaaggtcagccggttggcctaacttgggttttggccctttgtgcgaccaatgcctgctctgcataatggcgggcttacctctcccctgcttcttcatccgtgatggggattgctgctggttccttttgggccgtgtggaaacacagtgaccctccctgtccctgttgccccatagttcctggcccagaacgaagagaagccgtatgtggttgcccacttccacgaatggttggctggcgttggtctgtgtctgtgccgtgcccggcgcttgccggtggcaaccatcttcaccactcatgccacgctgctggggcgctacctgtgtgctggcgctgtggacttctacaacaacctggagaatgtgagctgcgattgtattgtgagacagcccaaagccctggcagggaatcgtgctcctaagagtgcaggctcccagcccttccaccatcagacatgcccccccccccccccatcctggccatcctccctcagacctgcctctcagagcgcagctgccaatcccaacctccctaagaccccagtccagtccctagtcttaccctggtccttttaacgctgacttttctcccttcctacccagttcaatgtagacaaggaagcaggagagaggcagatctatcaccggtactgcatggagcgtgcagcagctcactgtgcccatgtcttcactaccgtatcccagatcaccgcaatcgaggctcaacacctccttaagagaaaaccaggtagggtctgagctgtggtccctacatgtggctgaggtaagaggctctatgtcacactcaacaccaatgccattttactccagagagtagaacaacttccctaagtcctggattgagatggtaactcgtaaatgaagctggctgactctgtatcctgaaggataataggagctatagtctcattcatcctaattttatgagtaactaatataaatactgatcttctgaaagaaatgcaaatatggcgggctttatttttgaataaagtggaactagctaggaacttggtttactgatcctgcctcacgtgctcctaagtcttggccctaggtcctgatgagggttgatttgttgtaatcgtgcgtgcgtgtgtgtgtgtgtgtgtgtgtgtgtgtgcgtgtgcgtgtgcgtgtgtgtgcgcgtgcgtgtgcgtgtgtgtgtgcgcgtgtgtgtgtgcgcgtgcgcgtgtgtgcgcgtgtgtgtgtgcgtgtgtgtgtgtgtgcgtgtgtgtgtgtgcgtgtgtgtgtgtacgtgtgcgtgtgtgtgtgcgtgcgtgtgcgtgtgtgcgcgtgtgtgcgtgtgcgtgcgtgtgtgtgtgtgtgtgtgtgtgtatgtgtgtgtgtgtgtagaaaccagaggagaatgtagggggtcttctctctgtgactctccacctaattttttaaagaggtgtttgttatttttatttatgtatatgtgtgtgtgcgcgcgcgcgcatccttgtgtgggtacatgcacacatgcatggtgtgtatgtgcatgtgtggtgtgtgtgtgttcacagggggcggaagagggcttatatcctaatcttcagctgcagttacaagtactgttaaccaccccgtctgcgtgctgcaagagcagcagtaagctcatttgactgcagagccggttcatcagacccaacagctcttgtaagacagggtgtctcattgaacctgaagttcaccattttggtgaggcctcgtaagctcccagggcccacctctcttcccccacccccacactaggattataggcgaatttagccatgcccagcttttattgaatgtggtaggtctgatcttgggtctttttttaaataaactttataaacgcttctattttcatatgcattactctccatacctcagctcctagagatgggtagtgccatggatagtgccatgggtagtgccatggatagtgccatggatagtgccatggggcctgatatactcaggaactctgggaggtgcagttttccatctgtccttctcccattctgacacagggactgaaaataccactgctctgcttctccaggatatccactcccctctctctctctccctctccctctccctctctccctctctctccctctctctccctctctctctctctctctctctctctctctgagccagggtctcactatgtagctcagggtcatcttgattaacacgctctgtcatacttggctcacagtgcaccatctgtctttatgtctcttttagggactcaaccctttctcccctcatatacttttaaaaaatatttgtttttatgtatgtgtgtgcgtgtgtatggcacgcatgtgtgtgtgcatgtgtgcgagtctgagtgtataaacactggttacttttctgggaaatccaggtttaattctgagaacacacatggtggcttataaccatctgtgtgttccaattcctggagacctgaccacctcttcttgcctccacaggcaacaggcacatatatgttacacagtcatgcatgcaggcaaaaccaccatacacataaaatacaagaaaaaaatctaaaacagagtatgaagggtcaagtagcggtgttgaatcagtcccccgtggagctgagatcactaaagggccattataaaactgtgacattatgaaagggaattcagccgggcgtggtggtgcacgcctttaatcccagcattctggaggcaggcggatttctgagtttgaggccagccagggctacacagagaaaccctgtctcgaaaaacaaacaaacaaacaaaaaaaaaaaaaagaaagaaagggaagtcacctccccttcggtttttgcctgtgaagttgatcacaataaagaaatgaagcatggggctggtgagatggctcagtgggtaagagcacccgactgctcttctgaaggtccgaagttcaaatcccagcaaccacatggtggctcacaaccatccgtaatgagatctgactccctcttctggtgtgtctgaagacagctacagtgtacttacatataataaataaataaatcttaaaaaaaaaaaaaagaaagaaatgaagcatgggcccggcctgtctctcccaatcctagatattgtgacccccaacgggctgaatgtgaagaagttctctgctatgcacgaattccagaaccttcatgctcagagcaaagcacgaatccaggaatttgtgcgtggccatttttatgggtatgtgagccagacagtgaagagaggtggatgggggcgggggagaggtgagattcctgcgtctgaggaggagggctgtggttaggaccctgagctctgcggagagaagactcccgagtctcagggaagagagtgaggattccttggtcctcagtcctggacagcaatggtctccattatttcctgatatctggagccatgggatgggtattagggtaaatactcatctaaggagaaattctgcaattgcatgcatccctctgatacctgtgcactataaaaacttcctttggctctgtgacttctggggtttgatggcttcagcacagggttaggcgcttgggcctcacactcactcccctgtggcttctttaggcacctggacttcaacctagacaagactttgtatttctttatcgctggccgctatgagttttccaacaagggagctgatgtgttcctggaggcattggcccggctcaactatctgctcagagtaaggcctgctcggtgggacagtgggtggggagccggagggggaagtgtgtgcctagatggggagaacagagacccgagagatggctgtggaaacccttggcagagtatagagtcccagaaagatcagaagtgagatctggtggcctgtacctgttatcccagcacctgggaggctgaagcagaaggatcaaaacttcaaggtcagcttttgtctatagaatgagttcaaggttgacctgggcaattttgagacgctgtatcaaaataaaaaccagtctggcagtgtggctcagtggtagatcccctgcctagaatcgcccagtgagggggcaggagagtggctcagtggtaaagcgcctacgtattctagagatttgattttaatcccctatattattatgaaaaagaaaatcggaagtcccatcattcttagcagcaaaggcatgattgagtccacaaccctgatgtccctctcatccccgtccccaggtgaatggcagtgagcaaacagttgtcgcattcttcatcatgccggcccggaccaataatttcaacgtggaaaccctgaagggccaagccgtgcgcaaacaactatggtcagcaatccttgtccaggaccctgtgggcttcgtacctgcagtcgccatatcttatctctctctggctatctctgacatgatcaggattcctagtgaactcctctcttaaagcatgtggcggggcagacaagtgtgagcgcgtaatcagggcagccatcacacagtgaggtgacctgactccttgttatcttgctgaaccaccgaatcctcacggagcctgtttcctgctctctgaaacccacccgttcactgcccaccaggctctgaatatctaccacgaggcaggcactgttctaagccctgggaacagcagatgcatgtttaagaaatgaggaagagctgaaaatgtagctcatggtagggcgttcagcactcacaagccccggcacggcataaagtgggtgtgatggtccatttctcttaattctaatacttggggagtggaggctggagggtcaaaaggtcaaggtcatactgggcagcatactgagccaggaggcagttagagagagagagaagagactggtgtggttgggtcggcatgtccctgggaggatcgtaagtcatgagacagtagggtgacagtctttccgaacttgtgtgccagggtgttcctctcagaatattattcctcctgtttataagacagggcctagcagtgctctgtccaagactcattctaaccctaataatagctgacatttctggaaggctctctgtgtgctgggctttccctgacctctcggactcctctgcaaccacctgaggttgactctatttttacactcatctacaagaggagtcactgacgttggagaggaaaggccaccattcccagccacctcaaggaactcaagcagtctccaggtccccatcttccagtttgaggtcccccggggtgtggattctgaacccagacggaagtttctctggtgcccacaatgcctgacagaacatagtggctgtgatgtgacattgtttctctccgttccagggacacagccaatacagtcaaggagaaatttgggaggaagctctacgaatcccttttagtgtgagtgtcagcccctagctcttctcccatgattgccctgacccatcccgtcacttcattctgaaatgcctccccctccccttgcagggggagcctcccggacatgaacaagatgctggacaaggaggacttcactatgatgaagagagccatctttgccactcaggtatggtctttacccctgagcaaagtgctggccagttctggcatttgctgaggcctcttgctgcaaaggtttctgggagttctgttctctttggtggccctttacagagggtccagttctgaaatccagagaaataccgatagtaaaacacacccaccacatcacaaagggtaagatggtttattccggaaccaagtataagcatggattgggaacacagattccggttacagtgttccaacacagtgacgatcgtgtggctttttatgataacagaacactgacagtaatgaatgaaggccttgttaaaatacattgctggaggggataccagggggtccccttctcagaggagaaggggtgagggagagatgatgaggggagaatgggaggagagggggctgatattgggatgtaaagtgaacaaataaattaattttaaagagacactgctggggctggagagatggctcagtggttaagagcgccaactgctcttctgaaggtcgtgagttcaaatcccagcaaccacatggtggctcacaaccatccataataagatctgatgccctctcctggtgcgtctgaagacagctacagtatacttacatataaataaataaatcttttaaaaaaagagagagagactgctggagacatcaggctgagaaggggcacagaaagggggaacctcagctctatgtctgggggcatttccagtcttggtgaaaactagggtttctttttttttttttttttggtttttcgagacagggtttctctgtgtagccctggctgtcctggaactcactttgtagaccaggctggcctcgaactcagaaatcagcctgcctctgcctcccaagtgctgggattaaagaagcgtgcaccaccactgcccggcttaaaactagggtttctttaagttcctgcacccctaaaggttttaagtcacagcccttattagagacaggtagcaaagagtggcgggcgattcaggagcactgaactagaccaagatcaatttttgttgttgttgttgttggtggtgttttgttgttgttgtttgttggtttgttttgcctttgcagacagggtttctctgtgtagccctggctgtcctgaaactgtctctgtagaccaggttggctttgaactcagagatccacctgtttctgcctttggagtgctggtgtgtgtgccaccccacctgcccttatttttatgagacagagtctaattagactctctacctgccacattccagcatctccacagtcagtcagccttgcagaaggttgttctctgttaaggtacatttctttttggggacttcatagtttattgaacgttcagaagcccccaggtgacaacttctgtttctgctgcccctggggcgtgttaagggtagcagtttaaggggcctttaggagataggagtcaggcaagtcaatacacctagtgttttctggagaacactgaatgctcatcagccactattcgccgtcctagaactcactctgtagagcaggctagcctgtaactgagatctgcctacctctgctttccgagtaccaatattaaaggcatgcactaccaagcccagctccgtggctgtgtcttacctctcacagaaccctctgattcctggggttggagttaaaaggacaggaaacattagggagatgtggctcacaaaatgaaaactacaactcccatagtattttgtattatattgaccttggcccctgaggcttttgggagttagagttcttttttttttcctgtgggtcaaggggctggtgcctatgtttggcattaatgaggctgagttttgggatctagacagccagcatggattaatgaggtagaatgtgtttttgggcaaaatatttttgaagttaagcagccagctttactcatgaagaagccttttacatggggctgctgggagttgtagtttttcaccctcataccacccaggagtgggcccattaataacataatgatggaacagttgtaaagaacttgtcataaaagagagggctggggggctggagagatggctcagcagttaagagcattgactgctcttctgaaggtcctgagttcaaatcccagcaaccacatggtggctcataatcatctgtaatgaaatctgacgccctcttctggtgcgtctgaagacagctacagtgtacttacatataataaaaaattaattaaaaaaaaaagaagaagaaatgtcttttaaaaaaaaaaagagggggctgggatttcctgtctgtctttgtgttgcagcggtgacttctgaaattgtgcttaccttccctgagggggtagggcagtggggttggaagtgatggtggtgatggtagggtgtggattttcataagaaaactacaacttgggcctggagagatggcgcagtggttaagagcactggctgctttttcagaggtcaagagttcaattccagccgggcagtggtggctcacaaccatctgtaatgagatctgacgccctcctctgctgtgtctgaagacagagagtacttacatattaaaaaaaaaaaatcttttttaaaaaaaaaaaagaaaactacaactcccagctgtccttgtggcttgttatcaaatagacatagctatacacttggctggggtaggtacagttctttagagctccccacccccaactcctccccacccccccaccaccggcagaggggagggaggagtggagtcaacattctgatgtcattttggttcaagttacgtggttgcttccatcttggtaaggttctttgagtttttgttattgacgggcaagactatataactaaacacagacatttcctcacaagatgtggagtgcctctgtttagctgggatttttttgttggtttggtttagctttgcttgtgatagttaaaagtcgggctccagtcaggagtatgctgacctctgctggtagcttcttcagagccctgtgaccgccctccctcccctgacccctccctcatgtctcctgtctacagcggcagtctttcccaccagtgtgcacccacaacatgctggacgactcctcagaccccatcttgaccaccatccgccgaattggccttttcaacagcagtgccgaccgtgtgaaggtcagggctggcccgaggttggtggggtgtggctggggaaagggttcttggatatcatgcttctcctctacctagtctgtcaattgtactgtttagaaagaaccctggcttctgaagccaaatgaattcagtttaagtctttagcttgttgtggacttgctgtgtgtttctagagaaatgtaccctctctgggctactctaggtggctagaagtagctcagcccttctcctctgtcgacaggtgatttttcacccagaattcctttcttccacaagccctctcctccccgtggattatgaggaatttgtccgcggctgtcaccttggggtcttcccctcctactatgagccctggggctacacaccaggtgagtacaggaggtgaggggtggcggattgctgggtcctgttcagttctggcatgaacctgcaacacggttcctctgtttcaagatgatggtagtgacccatcctgtatccatgtgtcacatgtttgtctcacagaaggctacagagctcagactagatgactgatgttcatttccggtcgtcgacactgataacctcagctccaatttgctgatgttggagttgcacatgaaccaggccttccccggtgtctctcagacttgctatgtagcccagactggtcttgaactcctgatcttgtttccagattcggagtgctacaccactgtgatctgtttgtgcagtgcgggctgtagaagctggggctctgtgcctgccaggcaagccctctaccctctgagcttctcctcctggtccctagtgctgctcaacctcttcctctcctcacctctcttcagccctgagcttcgagttagaattgcttgtgtgtaattctccttcaccgagaggatcctgtggggcttttcctccttatttttgttttttaggcccgcttgttttctgatacccaagctggcctccagctcacaggtgtctgagggtgaccttgaactcttgacctccctgcctctacctttccttgtattctagacatattcttcaattctgcactcccgagcacacactcacacacacagacagacacactgacacacacacacacacagacacacatacacagacagacacactgacacacacacacatacacacacacacacactcacacacagacacacacacacacacacacacacacacactcacacacacacacagtaagttaattaagtttccttacagagcttgtgtgaggggtgaatgtgggaaggagatgttgggaggagtggctagaacctccagagaaggtctaacgtccccacccccacttcttttttagttcattattatgtttattcattcattttctaaatctttattaatttatttattgactttacatcccaattgctgctccccgccccgcccccactcctatgagggggacattaacaagcaggtcttggttttgagagtcacttgcaaatcattttgctctggttaaaatgataatagagccaggtggtggtggtgcacgcctttaattccagcacttgggaggaagaggcaggcaggtccatgagttcgaggtcagcctggtctacagagtgagttccaggacagccaggggtacatagagaaaccatgtctcagaaaaaaaaaaaaaaaaagttgataatgtttgggaaactaccatggggtctctgtctgccaagctgagcacctggcagtccttaccttagctccctgaaccttggaatcacacgctgctgccattgtatctcactcagatctttccttaccaccagtggacacgggctgtgagctgggtgtggaggcatactggggaagccgaggcaggagagttatctatcattccacaggctgactgagctacacagtaagaccctgtctcaaaagcaaaccaaccaaagagcatgtgcacctctaatgataactgatgggatgcagaggaggaagattaggagttcaaggtcatctttggctacatagtgggctcagggccatcctgagctacataagattctgtctttaaaaagggggtggggctggagagatggcttagcaactaagaatactgtgttctagaggacctagatttgctgggtatagttgcacatgcctttgatccctgtacccaggaggcagaggcaggtgggcctctgtgagttcaaggccaagcctggtctacacagtgggctccaggacagccagagctatatagtaagaccttgtctcaagaaagacaaaaataagggctggtgaggtggctcagccgttaagagaattgactgttcttccagaggtcctgagttcaattcccagcaaccccatggtggggttgtaatgggagctgataccctcttctggtgtgtgaagacagtgacagtgtgctcacatacataaaataaataaatcttttaaaacaaacaagccgggcatggtggtgcatgcctttaatcccagcactcgggaggcagaggcaggcggatttctgagttcgaggccagcctggtctacagagtgagttccaggacagccccagggctatacagagaaaccctgtctcaactccccccaaaacaaacaaacaaaaaaacaaagccaaaaataaaataaaacagccataaaaaacccagattcaattcctagcacccatatggctgctaacagtctaactgtacttccaggggatccaacactcttctggcctccacaggtgcttcatacacatggcgcatgtatgtaggcaaatcacccatacccataaaaattaaaaattaataaatctttgtttgtttgtttgttttgtttttgtttttgtttttggttttggtttatcaagacagggtttctctgtatagccctggctatcctggaactcactttgtagaccaggctggcctcgaactcagaaatctgcctgcctctgcctcccgagtgctgggattaaaggcatgcgccaccacgcccggcttcaaaattaataaatctttttttaaaagatttatttatttattatatgtaagtatactctagctgtcttcagacactccagaagacagcgccagatctcattacaggtggttgtgagccaccatatggttgctgggatttgaactcaggaccttcggaagagcagtcagtgctcttaaccgctgagccatctctccagccccaaaattaataaatcttaaaacaataactacaaccaacctcatgatcatgtcataatctaaagccgtgtcatgcacacacgagcacacacacagatacgaatcttaacgccgtgatggttaagtaaagttatttctgtgagccagcattggttagcgttccataaataagtatcctctttatcacaggtttccgcagattcacttttctaaggctctttggttcctagaaatgggggaggccccttgcgggtgtagccaggcgcggttggtttagttgccagcaggtggcgatgtagagccatctctaacagggcaggctactgagtagccttttttcccctcccacctttggttcctcagcggagtgcactgtcatgggcatccccagcatctccaccaacctctccggctttggctgctttatggaggaacacatcgcagatccctcagcttacggtcagtgcagagggaggacagaacatggcgggcgctggtccccatttcagactcttgttatcgttaacaaatcctgattcctgcctctgtaggcatttacattctggatcggaggttccgcagcctggatgattcatgctcacagctcacctccttcctgtacagcttctgccagcagagccggcgacagcgcatcatccagcggaaccgcacagaacggttgtcggacttgctagattggaagtacctgggccgggtatgactgatttcctttcctaggcctcctctggttctgtagccttaaagaattgagatagggctgggcgtggtgctgcatgctttaatctcagccctcagaaggcagaggcagatggatttctgtgagttcctgggcagcctgatctacagagggaatccaggacagctaggactctgttacacaaagaaaccctatcttgaaaaatcaaaaaagagagagaagatggagatagcctgatgtggtggtacagagctcaggagacagaggcaacctggatacaaagtaagttccaggacagccagagctacatagagaaacaaagaggctggaaatagatcaaagtttcctgcttattgtagttgttgtctctttaagcaagtgatgtgaggctgaagaagacagctcaatggttcaaatcccagcacccacatggcagctcataactatgtgtaactccaagatctgacactctcacacagacacacacactgtcaaaacaccaatgcacaaaacaaacaaacaaacaaacaaacaagcaaaaaagaaactgttgtcagagctagagacacagcattggctgctcttccagaagactagggttcagttccagcacccacatggtggctcacaaacatctgtaattccacttccagcagatttgatatcctcttggcttctgtgggtaccagacatacttctgatacctagaacacatgcagataaaacactcatacacacatatttttttttaaatcccaatatgatgatccctgtgattttagcactggaaggtaaagataagagaatacctggaggagctgtcgagatggcccagaggttaagagcactgactgttcttccagaggtcctgagttcaaatcccagcaaccacatggtggctcacaaccatctgtaatgagatctgatgccctcttatagtgtactcatacataataaataaataagtcttttaaaagaagaaaaagaggatacttggggccaggcagtggtggtacctagcactctggaagcaaatctctgagttcaaggccagcctgatctacatagtgagttcagtgacaggcaaggctagaaggcaagaagctctgtctcagcaaacaaataaataaaaaagaaagaaagagctaggcgtgttggcgcacgcctttaatcgcagcacttgagaggcagaggcaggtggatttctgagttcaaggccagcctggtctataaagtgagttccaggacagctaagactacacagagaaaccctgtctcgaaaaactgaaagaaagaaaggaaggaaggaaggaagaaagaaagaaagaaagaaagaaagaaagaaagaaagaaagaaagaaaaaaggggtacctgggattcactggacagccagcatcctatttatggcaagtccccgcctcaagaaacaaggtgaaagccaggtgtggggttgtctgcctttaatcccagcactttacaggcatagactgtgagtgcaaagccatcctagtctacatagcaagaccttgcctcaaggaaaaaaggaaacagagagagagacagacagacagacagagagacagagaagctggagagatggctcagtgtgtttaggaagagatcaggcagcactcacataacctggcatctcagaaacacttggaattccagctgcaaaagactgaatgtcatcttctagtccccacacacaggcacatgacagcacacatcatatgttcatatacataaataaatcttacaaaacaaaattcaggatgaattttgcctgagaaatgacatctgaagttgttctctggcctctgtatatagacacatatacacacacacatacatgcacatacatacacacatacatgcacatacatacgcacatacatacatcaaaaatgaaaagagagcaaggtgttagagtgcccaccttcagtctaagcacttggagacagagacaggagacaggtgggtctctgtgagtttgaggccagcttcttttacattgccaggtctgtaacagccaagagtacatagatccagtctcaaaaacaaaaaataaatacataaatgaataaataaataaaagataaaaaaatctagaaatagcatctggatagagttctcatttgtgtgaccaaaagagtatttgagaattcacctgccctgtaggaaggtatgactagagcatggtccatgcctagttcacttggaacattcacatttgcacaggaacatttgcataggagagatgagataactttgcccagtgtgctcatctgtataaaggtatagcaattggagcctggctcaaagttcaaagccagctaggggacacgtagtaacattcccaacgcaaaacagaaaatacaagaaggtgtggccatcggtacccaccagcccaggtttgtcatccagctacttggaaggatgaggcaggaaagtcaccagtgttaccaagtgaagattgctggggactccagcagtgacgggtggagagcatggctcagtggtggagcaggggagctgggcgtgcctttaggttttagacccttaactataatccaccagagaagtggttcagtggtagagaccctgcctcgtgtccaccagtaatgaggggtgtgtggctgggcttgtgtagaaaaaatgtggctggacattgatggccttgaataaatcccaagcactggggaggcagaggcaggcagatctctgtgattttgaggtcagcctgatctacagagttctatgacagccaggacttacacaaagaaactctcaaaaagcaaaccagacaaacaaacaaacaaacaaaaaagtgctgggggtttatctgcctgaagccaggtgtgtagcctctgtccttgacgctgtctgccctccctgtctagtactacatgtctgcgcgccacatggctctggccaaggcctttccagaccacttcacctatgaaccccatgaggtagatgcggtaagtggagccgggatcgacgctgggccaggagtgaagggctggggcaggctggtttggggttgacagttagtggacacctctagggacttaaatggcacccattcattttggtttgtgcctgctctgggtatgcagacccaggggtaccggtacccacgaccagcctccgtcccgccgtcgccctcactgtctcgacactccagcccacaccagagtgaggatgaggaagagccacgggatggacccctgggggaagacagtgagcgttatgatgaggaagaggaggctgccaaggaccgccgcaacatccgggcacctgagtggccacgcagggcctcctgttcctcctccacaggtggcagcaagagaagcaactcggtggacactgggccctccagctcactcagcacacccactgagcccctgagtcctaccagttccctgggtgaggagcgcaactaagctcccacccccatcccattccctgcctgtccagtgctcctctcgcagagggcctatgcagatgggagggtgcctgaaccccactccagactcttgagtgggacccctacccagtgtggtccatagcctaacctctgtttcagacactccagcccttgagctccaatcttggagttcccgcactccacgccgccgtgcctttcttggattgcaggatgcattctttgtgcactgatctggagtctccaggcttagactgggtcccagaggccaggcatctgccattgtttttcaatgccagaggttttaggacacctggtttattggcttccaggctgtggcttcttcgtttgatcctataatcatacagagtatgctttgctcaggcctgcctctgggaccacctcatgttggattctgtgtggcttcccgaatcagccaagttcagagttaggacatttcagggattaacataattgaaaatcagcctgcaaggtagctcagtagctctgtcgacagattgcttgtctagcatgcccgaagccctgggatctaactctagaacctcataaacctggtgcggtgatacacatctgtaatcccagcactcggtaggtagaggtagacggatcaagagttaaaggccatcatcctctgctacatagggagttcaaggccaaactgggcaacatgagacactgtctcaaaagcaaagtaaaggtggtggaatgctcacggtcctccatttcaacccacgactgcgatgctgggacatgctgcaaggttggcctccctgggtgtgttcttcaaaggagcatgcggagttggaccagacacctttctgccttttttctggaccagaccttcttttccttggtccagtgtcccctctagggaatgcctccattgagggcagaatgtctgtcaaccccacaagtgctcagcccactgtgaaaccactgggttctgggtcccagtggctgaatcaggagtcttttgtcactgtgctgcaccccggtcccctttcctgatacaaaaccgagcccaccggcttcttgaagccccacatgtacctcgaggcctttctgcctgcaagcttcagtgaatgggcgggcccctcctcacgtgtgctgtgtctggcccagtgcctttggtttgcatttgggagggggagggcagaaggtgtgtgattggagtgtgtctagagatgaaaaaaaaaaaaagaaaatacacctgtatttaagaatgccaactgtggctggacataagctcttgtggtgtggtgggtctctcttgcttccccagtcctgtattttagtttttacagataggatcttgccttgcccccccccccccagatggttcaaattcacagcagtcctatctcagcctctcaagagctggcattaccaggagtgccagcttcccccagattttggttctgctcttacgagctccatagacttctactctgtgcagatctgggttactgttctgaggtccccgtgctgttgttaggaaaggtacatctgactgcttagatttgagatccaagtggcaaacactggccccagtgaccttgggtagacggtggccgacatgggggacccttggggaaatcacaaatgcacaggtcaaaggacccaaagctcaaagcttacatggactagagcttctggggccagcttgagcaattggtgagatctagcctgaaaaagtgaaaagcccgggacagatgtccctaagcagaatgtccccgcaacacagtgagaaactggattccagccgcaggactttgttcctgcttctcaatgctgtttcccacgtggacatcgctgccagaatccctttagcaggttctccctgcttagcctcagtgacctgtgcatgactggagttactgctctaacccccaaccctcccgttatcaatctcatgcttttaggggtggagtggcctgactagaagaaacctgtagtgctgggtcatcttcctgtccacatgactgcttccccagagccatggcataatactgagccaagggcctatcatgtggcttcatacgacccttggacttgttttaaccagtgtgagctgacacagcagtattgtccggtgttccaccccccaccccaccaacttcccatgtttctaacccctttgcaatggtttagttcccatttgggcaatggtcccaagtttcacatcctctttttgatgggcccagcatctagttcggaatccagcaagacaccctggctccccaccctcgctcagccagcacccagggctcagcttgctcacctcctggccttctacttgtatgtttaactcatttctgaacttggatgtttaagccttttcacttccctgcacagtaacataaaccaaggactggcaaactgctacaggctacagactcctacgacctggatttgccacatgaaaaggcagatactaccacacagagatcgctgtaagttatcacatgtgggttcccacaaaaaacaggagtacatacagccaacctcttctttctcgtgcacccctcccacatatatagaaaaaacaccagctgctgtctcaaaaagctttatttttacttggtccaagacttgacagaggctccagggcggttacaaagctgcctagtggcttgagaggttcattcaggtggaggtcccagggcgggctggtgcagagcagaggggggagccccttggaaggtacagaggcctcctagtcgtgcttgagagtgaggcgctcaaagagatactcgcccagagacccctggggcgcgccagtctgcgctggttgtggccccgccaccctgcggaggttggtcagatggttgcccatcttcttgatgagtttcacctccttatccagatagtggctttccaggaagtcacagagctgtaaaggaaggaagagacatcagacaccgagttctaagagacaggtgtgagcaggaagctttaaaaaaaaaaaatcctgccctcaagggcaggtaggtaggtaggtaggtgtgtcctcagtactgtgtgtggtgcttctgcaatctgcactcgggaggcaggaggattaaattattgggagctgaaagccaacacacaacagtctaggttttgtcagagaagggaagaaatgattagcaaggtacttacatgagggtccgcgcgggcagaacccagggcatgcagatccaagagggcctgattcaggttcttctccatggccaaggcagcttccatggcctcctgggttttaccccattcatcttgagatggcttctgcacacaagaatatcaagcctagattacatccttcacccagccaatataaaactgactgcacaggaaaagtgggcacagtggggaaaaataatgggatactgtctttaatcccaccactgagagactgggcgagaagacaggaggatctcttgataggaggcaacctggtctacattgtgatttccatgctagctagagagacaccgcctctccccagcccagcagtcctcatctgtattctgtaattacccccattttccagtttctgacatcctggtgggtttagttgctctacatactcctattgtctacactagtcctgccaccactcccttccaagacgtctatccggatgcccaaactatggggatggcaagtaaacgcacatgctcggtagtgcccaatacccccgcttcttacctgcacatcctggaagagtgcacggcccccgcgatcgttctgaaactcgaggagacgctccgcgccctcgcgcttctcctcggccaattcgcggaagaagtggcctacgccctccagagccacgtcatcccgatcaaaaaagaagccctgcgggaggttacaaatacgagttaacggaacgcggtggctcggctcacactgtaaacttcaatcgcaacactcaagaggtagaggcagtacgtgtgagatttcgagggcagcccggtctgcccagcgagttccagtcagacaaaactactatgtacacagcgcgcagggagagggagaggtcggagcacagtctctgccctgcatggaacaaagaaggcccgcgcatgcgcaggagggtgggtgtgatggaccccccccccccccccccgttcagaaggcaaccccgagctgctcaccagagagaggtaggtgtaggaggcccgcaggtgcaagttgaccaggcggttcacggcagcttccacctcggtggaataattctgacgaatctgagaggtcatggctgatccggagtaggagctaaccgcgaagagacggtgcagactggtcctaggagctgaaggcagcgaggagatggtcccggaggctgcgactggagagacttgtaaaggcggctggaagcgagtacagtgggaatccccgggtctgttccgttcaaacactgttgaagcaagacacagatccacgggacctccaaggcgctgctctaagtcccgggcaaggagcgctccttttatagcatgttgaccctccggagggaggggtgccccaggcccgagactaaccaatcggcggtggcgtgaggcggagacggaaaagtatacttgaccaatccggagctgaggggaacctcgctccccacccaccca

SEQ ID NO: 2 - DNA, 3635 bp, Homo sapiens
  source 1..3635: mol_type = genomic DNA
tcctggcggctgcgaggtttcactgcaggggcgccagtgggctcagtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaaggtgggtggcatctacacggtgctgcagacgaaggcgaaggtgacaggggacgaatggggcgacaactacttcctggtggggccgtacacggagcagggcgtgaggacccaggtggaactgctggaggcccccaccccggccctgaagaggacactggattccatgaacagcaagggctgcaaggtgtatttcgggcgctggctgatcgagggaggccctctggtggtgctcctggacgtgggtgcctcagcttgggccctggagcgctggaagggagagctctgggatacctgcaacatcggagtgccgtggtacgaccgcgaggccaacgacgctgtcctctttggctttctgaccacctggttcctgggtgagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccagatattgtgacccccaatgggctgaatgtgaagaagttttctgccatgcatgagttccagaacctccatgctcagagcaaggctcgaatccaggagtttgtgcggggccatttttatgggcatctggacttcaacttggacaagaccttatacttctttatcgccggccgctatgagttctccaacaagggtgctgacgtcttcctggaggcattggctcggctcaactatctgctcagagtgaacggcagcgagcagacagtggttgccttcttcatcatgccagcgcggaccaacaatttcaacgtggaaaccctcaaaggccaagctgtgcgcaaacagctttgggacacggccaacacggtgaaggaaaagttcgggaggaagctttatgaatccttactggttgggagccttcccgacatgaacaagatgctggataaggaagacttcactatgatgaagagagccatctttgcaacgcagcggcagtctttcccccctgtgtgcacccacaatatgctggatgactcctcagaccccatcctgaccaccatccgccgaatcggcctcttcaatagcagtgccgacagggtgaaggtgattttccacccggagttcctctcctccacaagccccctgctccctgtggactatgaggagtttgtccgtggctgtcaccttggagtcttcccctcctactatgagccttggggctacacaccggctgagtgcacggttatgggaatccccagtatctccaccaatctctccggcttcggctgcttcatggaggaacacatcgcagacccctcagcttacggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccggtactatatgtctgcgcgccacatggcgctgtccaaggcctttccagagcacttcacctacgagcccaacgaggcggatgcggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaatcccaaaaaaaaaaaaaaaaaa

SEQ ID NO: 3 - DNA, 3443 bp, Homo sapiens
  source 1..3443: mol_type = genomic DNA
tcctggcggctgcgaggtttcactgcaggggcgccagtgggctcagtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaaggtgggtggcatctacacggtgctgcagacgaaggcgaaggtgacaggggacgaatggggcgacaactacttcctggtggggccgtacacggagcagggcgtgaggacccaggtggaactgctggaggcccccaccccggccctgaagaggacactggattccatgaacagcaagggctgcaagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccagatattgtgacccccaatgggctgaatgtgaagaagttttctgccatgcatgagttccagaacctccatgctcagagcaaggctcgaatccaggagtttgtgcggggccatttttatgggcatctggacttcaacttggacaagaccttatacttctttatcgccggccgctatgagttctccaacaagggtgctgacgtcttcctggaggcattggctcggctcaactatctgctcagagtgaacggcagcgagcagacagtggttgccttcttcatcatgccagcgcggaccaacaatttcaacgtggaaaccctcaaaggccaagctgtgcgcaaacagctttgggacacggccaacacggtgaaggaaaagttcgggaggaagctttatgaatccttactggttgggagccttcccgacatgaacaagatgctggataaggaagacttcactatgatgaagagagccatctttgcaacgcagcggcagtctttcccccctgtgtgcacccacaatatgctggatgactcctcagaccccatcctgaccaccatccgccgaatcggcctcttcaatagcagtgccgacagggtgaaggtgattttccacccggagttcctctcctccacaagccccctgctccctgtggactatgaggagtttgtccgtggctgtcaccttggagtcttcccctcctactatgagccttggggctacacaccggctgagtgcacggttatgggaatccccagtatctccaccaatctctccggcttcggctgcttcatggaggaacacatcgcagacccctcagcttacggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccggtactatatgtctgcgcgccacatggcgctgtccaaggcctttccagagcacttcacctacgagcccaacgaggcggatgcggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaatcccaaaaaaaaaaaaaaaaaa

SEQ ID NO: 4 - DNA, 3453 bp, Homo sapiens
  source 1..3453: mol_type = genomic DNA
tcctggcggctgcgaggtttcactgcaggggcgccagtgggctcagtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaagggtgtatttcgggcgctggctgatcgagggaggccctctggtggtgctcctggacgtgggtgcctcagcttgggccctggagcgctggaagggagagctctgggatacctgcaacatcggagtgccgtggtacgaccgcgaggccaacgacgctgtcctctttggctttctgaccacctggttcctgggtgagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccagatattgtgacccccaatgggctgaatgtgaagaagttttctgccatgcatgagttccagaacctccatgctcagagcaaggctcgaatccaggagtttgtgcggggccatttttatgggcatctggacttcaacttggacaagaccttatacttctttatcgccggccgctatgagttctccaacaagggtgctgacgtcttcctggaggcattggctcggctcaactatctgctcagagtgaacggcagcgagcagacagtggttgccttcttcatcatgccagcgcggaccaacaatttcaacgtggaaaccctcaaaggccaagctgtgcgcaaacagctttgggacacggccaacacggtgaaggaaaagttcgggaggaagctttatgaatccttactggttgggagccttcccgacatgaacaagatgctggataaggaagacttcactatgatgaagagagccatctttgcaacgcagcggcagtctttcccccctgtgtgcacccacaatatgctggatgactcctcagaccccatcctgaccaccatccgccgaatcggcctcttcaatagcagtgccgacagggtgaaggtgattttccacccggagttcctctcctccacaagccccctgctccctgtggactatgaggagtttgtccgtggctgtcaccttggagtcttcccctcctactatgagccttggggctacacaccggctgagtgcacggttatgggaatccccagtatctccaccaatctctccggcttcggctgcttcatggaggaacacatcgcagacccctcagcttacggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccggtactatatgtctgcgcgccacatggcgctgtccaaggcctttccagagcacttcacctacgagcccaacgaggcggatgcggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaatcccaaaaaaaaaaaaaaaaaa

SEQ ID NO: 5 - DNA, 1276 bp, Homo sapiens
  source 1..1276: mol_type = genomic DNA
agtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaagggtgtatttcgggcgctggctgatcgagggaggccctctggtggtgctcctggacgtgggtgcctcagcttgggccctggagcgctggaagggagagctctgggatacctgcaacatcggagtgccgtggtacgaccgcgaggccaacgacgctgtcctctttggctttctgaccacctggttcctgggtgagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccaggcatctggacttcaacttggacaagaccttatacttctttatcgccggccgctatgagttctccaacaagggtgctgacgtctttctggaggcattggctcggctcaactatctgctcagagtgaacggcagcgagcagacagtggttgccttcttcatcatgccagcgcggaccaacaatttcaacgtggaaaccctcaaaggccaagctgtgcgcaaacagctttgggacacggccaacacggtgaaggaaaagttcgggaggaagctttatgaatccttactggttgggagccttcccgacatgaacaagatgctggataaggaagacttcactatgatgaagagagccatctttgcaacgcagcggcagtctttcccccctgtgtgcacccacaatatgctggatgactcctcagaccccatcctgaccaccat

SEQ ID NO: 6 - DNA, 31000 bp, Homo sapiens
  source 1..31000: mol_type = genomic DNA
ttttttttttttttgagacacagccttactctgtcacccaggctggagtgcagtggcgcgatctcggctcactgcaaccttcgcctcccgggttcaagcaattctcctgcctcagcctccagagtagttgggactacaggcatgtgccaccacgcccggctaattttttttgtatttttaataaagacggggtttcaccatattagccaggctgctctcaaactcctgacctcatgacctgcccgcctcgaccccccaaagtgctgggattacaggtgtgagccaccgcacccggcctaattttcttatttttttgtagagacagggtttcgctatgttgccagggctagtcttaaattcctgggctcaagcaatccttctacctcggactcccaaagtgtgggattacaggcgtgagccactgtgcccagcccacaggtctttcttctcttcttcccctagagaggacatgcagttatccagcccagaggaagcctcctccctctggagccaatcgtttagactttctggccatcaatggtcacaagctttccctgtcactgtgtgaaccttcccacctaccgtctcctataggaagctggaacccaggagcatagaacattacccccagactacaagcagaagactaggcatccccaccccacaaaaaagacatgaatccaggaatcccaaatgtagctctctcaacataaacaggccccttaccaaggactgtctcctataaagactttctctttcatcataagtctctccaaaaccttgctgcccgctgttcccatgaaaaccaaggtcatttcaaaactcagcacacaccgtcccctcatacttccaatactttctccacggtagttctggtcatagccttcctcttctacttccctatccacctcctcctcctccttgccctggtgacagcaacagactcaaatctcatccccagctaccaacaactttctccccctcccttcctccatcttgactgggctcatcatggaaattgaggcacctgtaagcccagcccagtccctaagttcaggtcacattacctttttttttttggagacagagtctcgctttgtcgcccaggctggaatgcagtggcgcgatcttggctcactgcaacttctgcctcccaggttcaagcgattctcctgcctcagccttctgagtagctgggattacaggcctgcaccaccacacctggctaatttttgtatttttaatagagacggggtttcgcaatgttggccacgaactcctgacctgaagtgatccgcccgcctcggcctcccaaagtgctgggattacaggcatgagccaccacgccaggccaaaatcgcttctttattcaggtaattgagcaggtgccaggccaagtttcctggcctctccttcattcacatgtattcattcattcaaatattcaagtgataatttattgagcatttacactgagacgtagcccgtgtgaaatactggaaatatgaaaatggccaaaacggtagcatgacaagatcccgatttctgggtccctaatccttcagtcctctccactaagactctcttgttcctcgacaatttcttcccactaaaggctcactaggcgccctcgcgctctcagacacactgaacaggtacctgcctgtgcttccagcttggaactcaagtgccattggcaccactaacaatccaaggctcccaaatggctcgaagcgcaaggtcctacaaccctcctcatcacacactggcgccaaggactgcggaaccggccgttgcgccaagatgattttgaaagttggagttcctatagcatgatggggccagacccgagattctgggatcccagccccctccccgcctcagatccagaagtccagcccccatgtccccttctcccccagatctgggtggccgagcttcctcctctttcagacccaggagtctgaacccccagatcccccctccctcagacccaggagtcagggcctccagcatcctcctctctcaaactcgagactgctagctcgctcctcaccctccatgcacccacacttacaacggttgccatgatgctcaccaactgccagagtcctagcggaaacggaaacgcgcgaggagcgagatgttctggctcctctaggctagctggttcttcaaagatctcgatggttcctcataaatatgtaaatattgtgggggcgtggcgggactttatgccccgccctcccaccgaggctgcgctggaagacgggcgtccggaactcctgggtccattcccaatgtctcctggctcctcctccttttgctcagccccaccaccgccccgctcaggccgattgcaaaattcttagctgttctttgagtaaccacacgtcctccacgctcctttcctctttatcccaccctcctcctcctccccactttgaggcctccagctccagattgacagagctgaaggccactcgtttccgtaaagcgcagcggcaagctccgccccctggaggtctcacggccgaggcggggccgggacacagcgactccaacccgttccaccaatgaggacacgcggaaatccccgcccccgggcgtgggccaatggcgctggctggggggggcggccaccgcctcctggcggctgcgaggtttcactgcaggggcgccagtgggctcagtgacgctgcggcctccttctgcctaggtcccaacgcttcggggcaggggtgcggtcttgcaataggaagccgagcgtcttgcaagcttcccgtcgggcaccagctactcggccccgcaccctacctggtgcattccctagacacctccggggtccctacctggagatccccggagccccccttcctgcgccagccatgcctttaaaccgcactttgtccatgtcctcactgccaggactggaggactgggaggatgaattcgacctggagaacgcagtgctcttcgaagtggcctgggaggtggctaacaagggtgagcacgccaggcgtcggggcccttgacgagaaggacagtagtggatgggggcccggaactctgagttgtaggaggacggggctactaggggaacccttgtgacctgggcaaggaggctgtgtccttgaggaggggctgggaagccggaggtgggtgggagagggatggttcgtttgacttaagatgaaaggaggctggaagcccgggttctgggtcctagggaagagggcgctaaaatgagactcctaggtcctggaggcaggcttccgagagactagatacctgggtcctgaacagggaaggggatggggggatgggatttctgcgtgcaaacctgggaagctgccggggtttcagacctatgaccctaacagcatggtctaggggataaaactccagatttgagagagagctgactgggagataggaggcctgagcggcggtgggaggacctgaggatctggctgcttaagaataaagatgggacgggctggagactgtaatgaatgcctgggctttgaaggagctgagggcctgagctattagttcctaggagtagaggagcccatggcctgggacacatgcgtactggaataggggaaggctgggaagtcctaagttagtcagaggctgggccctaggatgggaggggctaggagcctgcatttctgtgtcttgggcttcagaaagctgaggttctggccttggagaaggctggggctgaaatccccaggaacaagccaatctcacacctcaaagagcagggtctggggggttgctttgctgggtcttggaggagaaagggactgtgtctcccaggaggagagggggcccgtgaacacccagccgggtatcgccagggcctgggctccaggatgggaggaggctgggggctggggctcctgggtctgggatagggtcatgggagaggcagagccctgcatgctggtgtccctagtcagggagaggaaattgggaggcaggtcagcaaggtccctgggggcagggaatgaccatgcaggcttctcttgggcaatggtgagaagctggattggccatgagggggtctccctcactgggttttggagcctggctgcgatttaaacccagctcctgtgagctgggaactcgggaacctgagtccgggtagtcagtcctcagctctgccatccttagagccccagtgcctaattccttcagaccaaggggtcccggcacccagccccttctctctcagacccagaagtctagcctgagccccctcctccttcagggtcctgaccccagcccccttctcccttagacccaggaggactgccctccagccctcgtgttcccctcatccaagcttccaacccctactccaaactatgggctcccagtcccctcttccccctgagctagaggtctggggcctgagtgccccctacatccccggcccaccccaccccaggccccagaactatggacctcggccggggcctgccctcacacgcttgggcccacagtgggtggcatctacacggtgctgcagacgaaggcgaaggtgacaggggacgaatggggcgacaactacttcctggtggggccgtacacggagcagggcgtgaggacccaggtggaactgctggaggcccccaccccggccctgaagaggacactggattccatgaacagcaagggctgcaaggtgggacgtggcccagcccagggcagaagcgggtggacagcctgccatcggggtgggagccacagacggagagggtgcagggtggacagccccagccagagagcgaggcaggcttggacacaggaaggaggcgtgataggacacccagacagatgcagggcctggaagtgggtgggtggatcccagacatgggcgaatggacagacagatggcaagtatgagacagactgagacggagatccaagaggccaaggtcctcagacccagagagaaagagagcaagatcgaaagacagcctggtcaacatggcgaaaccccgtctctaccaaaaatacaaaaattagctgggtgtggtggcgggtgcctgtaatcccagctactcgggaggctgaggcaggagaatcgcttgaacccgggaggcggaggttgcagtgagctgcgattgggccactgcactccagcctgggcgacggagcgagactctgtctcagaaaagaatgccagccagccaggcagagctggggagaagtgggcctgaggtcaggaaggagcaggattctggagcttgaagttcaagttcaccctccacttgctgcctgtgtgtccttggctaatggacatgctaatagcgtctaccttctagcggtgctgggaggaggaaacagttaataaggacaaagtggttagtagagttcttggcatgaaggtagcacattgtgtgggcggtgattgtcattgttatgatcatcgtcattattgttactaacagccaagcttggagagtgagggggacaggcagaccaagggagaagggtggatggagccacaggcaagagtaatagactggggacagaagagaggccaccagatgtttcagaaagccagtcctagaaggagagagaagcatggaatatcggggaagagaggagaggtcagtggacagacccacaggacagagtctgagatgggcagatcagggattagagaggagggggagttccagggccccagggtgaagacggaaagagacacgctgttgtccaaggagaaagccatcaggcggcacgctgttgtccaaggagaaagccatcaggcggcagggctaagggacagacaggtccaggctggggatggagaggcactgtggttcctttactcattcacagcctctggtttggggtggggtctggaatccattcaggtccgactgtgtccctaagggaacccagaacagcccagtcgcttcctgagggcagctgcttcccacgtgctgggggcctaagaatagcccccccccccccccagcaaaagggcagacaacagctgggagggggagggacctcagagcgaggctccaggcagcaaaaagccaggtcggcgaggagcggggctgcttctccactgctgtggcagcaggcccagaaccaaagccatggtctcagggaggaaaacagccaactggagaaatgtggcagaggcagaagtgagacagcaagtcttagaaacacggcagtggctagaaaaatgcagccctggggcacggacagaggatgggctatgttagaaatgtgtggcgtggccgagggcggtggctcatgcctggaatcccagcactttgggaggccgaggtgagaggatagcttgagcccaggagttcctgacccacctgggcaacatggcgaaactctgtctctactaaaaatacaaaaaattagccaggcatgatggtgcgtgctcgtgatcccagttactcaggaggctgaggtgggaggatgggatgtggaggctgtagtgagccataattttttttttttttttttcagatagaatctcactctgtcgctgagtctggagtgcagtggtacaatctcgactcactgcaacctccgcctcccgcgttcaagcaattctcctgcctcggcctcccaagtagctgggacctcaggcacatgctgccacgcccggctaattttttgtattttagtagagacagggtttcaccatgttgcccaggctggtctcaaactcctgagctcaggcaatccgcccgcctcggccccccaaagagctaggattacaggcgtgagccaccgcgcccagcccagtgagccatgattgcaccactgcactccagcctgggtgaccggagtgagaccctgtcaaaaacaaaaacaaacaaaaaaaaatggaagaaagaaagaaaaaggagagagggagggaaggagggagagagggaaggaaggaaggaaggaagcaaggaaggaaggaaggaggctgtggactacaaaatgggcctatggatagaggaaagaggtcgcaggtgacagatatgtgatgaaaggaacagaaacaaggcatcatggggaagaaaagtggcagcatgaacagtaagagaatcattacaggctgggtgcagtagctcacgcctgtaatcccagcactttggtaggctgaggccaggagtttgagaccagcctgggcaacatggtgaaaccctgtccttacaaaaaagttaaaaattagccgggatgtgataccttgtgcctgtggtcccagctacgtgggaagctgcggtggaaggattgcttgagcctgggagatcgaagcttcagtgaaccgtaattgcaccactcccttccaggctggaggacagagcaagaccccgtctctgaaaataaaaaagggcctgctttaggtggctcacacttctaatctcaacactttgggaggctaagcaagaaaactgcttgaacgcaggagttcacgatcagcctgggcaacatagtgagaccccatctccacaaaaaattaaaaaatcagcctggcatggtggcccacgcctgtaggaggtgaggtgggaggattgactgagccagggaggttgaggctatagtgaaccatgttcacaccactgcactccagcctgggcaacagagcgagaccctgtctgaaaacaaaaaagaagcatcccgtggaaggaattagcatcagaaagaaaagacagtgggagatgcagatggttggtaagagaaatgtggcagcaagtacagaaacaaggcagcaggtaggggaaatgatttgcagagtggccagacagggcagtgggggcatagacacaggacatgtgacatatgtggttataagcagagaaaggaggcagcgagtgggagaagggtagcagttacaggaattaaacaggaggctgggtgcggtggctcacgcctgtaatcccagcactttgggacgccgaggcgggcggatcacaaggtcgggaaatggagatcatcctggctaacacagtgaaacctcgtctctactgaaaatacaaaaaaaagtagccgggcgtggtggcgggccgctgtagtcccagctactcgggaggctgaggcaggagaatggtgtgaacccaggaggcagaggttgcagtgagccgagatcatgccacggcactccagcctgggcaacagagccagactccgtctcaagaaaaaaaaaaaattagccaggtgtgggagcgggtgcctgtaatcccagctacttgggaggctgaggcaggagaattgcttgaacccgggagacagaggttgcagtgagctgagatcgcaccactgcactccagcctgggccacagagtgagactccatctcaaaaaaaagaaagaaagaaagtaagcagggggcacggaaaatattgcaacagagatggaaaaaatgtgtagcaggaataaatcatgcaaatagagaagtaaggctaaggcagcaggtgagaaattagcagcagatatggaaacagaagataagagagctgaggcttaagtctatagacatctgcctgcaaatgggagaaccacggatgagtgaagatatgaggctgaagcccagagcctccctatggtgcctccccaaccccatcccaggtgtatttcgggcgctggctgatcgagggaggccctctggtggtgctcctggacgtgggtgcctcagcttgggccctggagcgctggaagggagagctctgggatacctgcaacatcggagtgccgtggtacgaccgcgaggccaacgacgctgtcctctttggctttctgaccacctggttcctgggtgaggtaggccccgtctgacacattccacccatacctgaaactacaatgaccatcagcccctgggccagaggatgggcttctccctgggcattctgggagtcatagggtcctttaatccaaggattagctctggcctttgaggacatgaacatccaagggcccaggctctactctcgatgccaacttcatttactctagtcttccaagcatagggattcttggaatcagcctcaaagtgaggctggcgggcttgcccgaggaattctaggagttgtagttctctagtgtgatattgctggactcagctctcagatgctgccctggatgctcatgaatccccagcctgacctggttttgacccccttctaggttttaagaggctcaagaagcttagtttccaggctgggcacggtggctcatgcctgtaatcccagcactttgggaggccgatgggggaagatcacgtgaggccaggagttcaagatggccaaacctcgtctttactaaacatacaaaaaaattagctgggtgtggtggtgggcacctgtaatcccaaatcccagctacttgggaggctgagacaggagaattgcttgaacctgggaggcgggaattgcagtgagctgagattgcatcacggcactccagcctgggccacagagtgagacactgtctcaaaaaaaaaaagaaaaagaaaaaaaaaaaaagaagcttaatttctcatgccctgggacaaataattgaggaactggctgctcctatggcattctgggagttgtagtccctttgccaatgttgggtgcagacagcgtacccttgagacaaggggtctttggaaactatgtccaggacctgggaacatgaaaacttcagtctgtccaatctaaccctgccaggacttagatggttggccaaaaaatcctcagttcctgtcaacccctgaggaggcagtcacttgagcccaggagttcaagaccagcctgggctgcctcagggatttacaggtccctgtccggttgggattgctgcagtgggccgctgcccactctgtgcagtagtggtgacagattgacctctccccgagtccttgtccctactggggattgccgattcttttcccaacactcgtggagacagtggccctgtccctgttgcccacagttcctggcacagagtgaggagaagccacatgtggttgctcacttccatgagtggttggcaggcgttggactctgcctgtgtcgtgcccggcgactgcctgtagcaaccatcttcaccacccatgccacgctgctggggcgctacctgtgtgccggtgccgtggacttctacaacaacctggagaacgtgagctgggaccgtggcagatgggggactgcgagtatgccaggaaaacccaaagctctggggggcccaaggggatggcaccccctcttctcccaggatccaggagtccaagtccccagacccctcctacctcagacccaggaatccagcccccagcaccaatttctcttgggaccccagtgtctggacctccagagtcttctccccaatccttctaaccggactgctgtcccctcccacccagttcaacgtggacaaggaagcaggggagaggcagatctaccaccgatactgcatggaaagggcggcagcccactgcgctcacgtcttcactactgtgtcccagatcaccgccatcgaggcacagcacttgctcaagaggaaaccaggtaggggctgggctgaagtccccagacgtgaatgaggtaggcagctgtctatgccaaagccagttgctcctcagcccaagaagcccactgtaagttgaaatagcataaaccgaaaacgtatttcggctgggcatggtggctcacacctgtaatctcagcactttgagaggccaaggcaggagaaccacttgagcccgggaatttgagaccagcctgggcaacatggcgaatccctgtctaaaatattcgccagacatggtgtgtgcctgtatccctagctactcaggaggctgaggtgggaggattgctcgagcctaggaggtcaagggtgcagtgagctgtgatcgtgccattgcatgatttgggtggcctggagagaccctgtcacagcctgggtgacacagtgagaccctgtctcaaaaaataaaaaaatgcatttaatacacctaacctactgaacatcatagcttaggctagtataccttaaatgtgcccagaacactttatgttagcctacgactgggcaaaatcatccaatacaaatctgttttattttatttttatttatttatttatttttgagacggagtctcgctctgtcgcccaggctggagtgcagtggcgctatcttggctcactgtaagctccgcctcccaggttcatgccattctcctgcctcagcctcccaagtagctgggactatacgcgcctgccaccacgcctggctaattttaatacaaacctattttataataaagtgttgaataggctgggcgcagtggctcacacctgtaatcccaacactttgggaggccaaggcgggcagatcacgaggtcaggagatcgagaccagcctggccaacatggcgaaactccatctctactaaaaatacaaaaattagccgggcgtggtggtgggtgcctgtaatcccagctactcgggaggctgaggcaggagaatcacttgaacccaggaggcggaggttgcagtgggccgagatcacaccactgcactccagcctggacaacagagcgagactccatctcaaaaaattaaaaaaaaaaagtgttgaatatctcatcaaaatccaaaattggactcatgtggatgtatgtaactttcacaccatcataaggttgaaaaatcataacttggctgggcacggtggcttacacctgtaatcccagcactttggaaggctgaggtgggcagatcacctgaggtcatgtgctcaagaccagcctggccaacattgtgagaccccctctctgccaaaaatacaaaaattaggtgggcatggtggcaggcgcctgtaatcccagccacccagctactcgggaggctgaggcaggagaatctcttgaacccaggaggcagaggttgtcgtgagccaagattgtgccattgcactccagcctgggcaacaagagcgaaactctgtctcgaaaagaaaaatcataacttggaccatcctaagtcctaagctggggaccatcagttaccatttttttacctaaagaatagagaactacagctcccagaaggctgtgggggtggtgacctgtatttgaggctctctgactatatactctggaggctaatgggaggtataatttctttcatattaatttcagatatgataaaattctgtaattggctaatgtagattggtcttatgaaagggaatggaagatcagtggactttgcttttgagaaaggttaggtcagaactgggcattcttcccagcccttagtatccgttctcagtgagtataaggtgagaactacaactcccagaaggcctttgactggttagctctatatgttccagaactttgccctaggcgctgatgggggttgtagtttttcgttgatatactttaatttctatgaggagaagaaatgattgggtccctgtaagaagcttggaagtagaggatatatcctaggagaagcaggactttggggatgtttaatcatttccatctcagggtgggctaagaggaaacaaaagaagtacaactttcccagaatcaccagtgtggatcagatccctgttagccatttgcagtagctgaggagacttatacttgtgcttgtgaacctaagcaagtaatattaggcaagaaggcatcttggagtcagggtaggtgtgtcaccaagggaactggccaggcatggtggctcacacctgtaatcccagcactttaggaggccagggcaggaagactgcttgaagccaggagtttgagatcagcttgggtaacatgacaagaccccatcactacaaaagtaaaataaataaattagccgggtgtggtagtgcacgcctgtagtgccagctactcgggaggctgaggagggaggattgcttgagcccaggaggtcagggatgcaatgagctgtgattgtgccactgctctccagcctgagcgacagagagaaaccctgtctccaaaaacaaacaaacaaacaaaaaaggtgggaggggggacctcataggaacgctcttgaaacaaaaaaattgcaaaaggcaaagggtgttttcttcatttattcattcaaccacaacattgatctgaatgaacaaataaaacatgggaccctcacccccaatcccagatattgtgacccccaatgggctgaatgtgaagaagttttctgccatgcatgagttccagaacctccatgctcagagcaaggctcgaatccaggagtttgtgcggggccatttttatgggtacgtggggcatatacctaggtcttaagagaggagggcgttgggagcctaaccatctaggtctgggggagggggcagctgggggcccagactcctgggtctgagggagggggcacctgggtattcggcctccttaattcttgagcgctggaacaatgggctcaggattaaaggaaatctccatctaaagagaagaactacaactcccaggaggccccgtgatgcctctgtattacctaagacatcctctgcccccccacccatagcttctgggatttgtagttttgaggccagggctagcgggtgagggctttgggctttaccgtgccttgtgggttctttaggcatctggacttcaacttggacaagaccttatacttctttatcgccggccgctatgagttctccaacaagggtgctgacgtcttcctggaggcattggctcggctcaactatctgctcagagtgaggcctgggctatgaggggacagggagggcaagcaaagcagtttataactagggagaacagccaggcacagtggctcacacctgtaatcccaacacttttggaggccaaggcgggaggatcacttgagcccaggagatggagaccagcctgggcaacatggtgaaaccccatctctacaaaaaatacaaaaattagccaggcatggtggctcacacgtgtagtcccagctactcgggagggtgaacggggaggatcacgcgagcccgggaggtggaggttgcagtgagccaagatcatatcactgcactccagcctgggcgacagaaaaagacgctgtctcaaaaacaaaacaaaacaaaacaaaacaaaaaattacagaacagaaacccaagaagccaggaagatgggggtaggaaagcccctgagtatggtggggggccgtgaggggagtatagagacccagaaagaaggtcagagtccaggtggggactgtcctataattctccttgacaggaatgggtatggcaggatcccagtggtcttgacagaaaggctggctggttccaggcttcccccacatgatcctggcctccccctcctttctttctgcgcaggtgaacggcagcgagcagacagtggttgccttcttcatcatgccagcgcggaccaacaatttcaacgtggaaaccctcaaaggccaagctgtgcgcaaacagctttggttagcagccctcgctccggcccctggcgcagcccactcccagatgcatgctttgggtcctgtgtttctcctagagtctcagttttttgttttgtttgtttgtttgtttgtttgtttttgagacagtctcactctgtcgcccaggctggagtgtagtggtgcgatcttggctcactgcaacctccacctcctgggttcaagcaagtcatctgcctcagcctctcgagtagctgggactacaggtgcacaccaccatgcctggctaatttttgtattttcagtagagatggggtttcaccacattggccagactagtctcaaactcctgatctcgtgatctgcccgcctcagcctcccaaagtgctggaattacaggcatgagccaccgcacccggccctcttttttttttttattttgagatggagttttgctctgtcgcccaagctggagtgcaatggcatgatctcggctcactgcaacctctgcctcctgggttcaagcgattttcctgcctcagcctctgagtagctgggactacaggggtgtgccaccacacctggctaactttttatttttattagagacaggttttcactatattggccaggctggtcttgaactcctgacctcaggtgttccgcctgcctcagcctcccaaagtgctgggattccaggcgtgagccaccacacctagcctttttttgtatttttagtagagatggggtttcaccatgttggccaggctgatcttgaacacctgacctcaggtgatccagccgccttggcatcccaaagtgctgggattacaggggtgagccactgcgcctggcttagtttctttttcttttctctgatttttttttttttttgaaacagagtctcactgttgcccaggctggagtgcagtggcgtaatctcagctcactgtaacctccgcctcccagcaaagctcagtttcttcatctgtaaaaccgttcattcattagcagagatactcactgagcatctactatgtgctagctaatttttatatttttagcaccgatgggatttcaccacgttgaccaggctggtcttgaactcctgacctcaagtgacctgccctcctcatgtattccaggtcctgggaatacagctgtgaaccagaaagataagatccctttcctcatggagcttgtattctagaaaatcctgtagacggaatttacttttgggcaaaaataaagcagaactggaatatgagacggggttgtcaggacaggccacactgagaaagtgttattgggcagagacgaggaaaggtgacgagggagcaagccctgtgggtatctgaggaacagcaaactcgaaggccccgaggcatgattgagacgcggtggggagtcccatggggctggagtgacatgtctcatagagaaggggtagaaagtgaggcaagatggagggcacctgtagtgccagctacaggaggcaggaggatcattttggccctggaggttgaggctgtagtgaggcatgattgcgccactgcactccagcctaggcaacagagtgagactctgtctcaaaaaaataaaaaagaagtcgggcgtggtggctcacacctgtaatcccggcactttgggaggccgaggcgggtggatcacctgaggtcgggagtttgagaccagcttgaccaacatggtgaaaccccgtctttcctaaaaataaaaaaaatcagcagggcgtggtgacacacatctgtagtcctagctactcgagaggctaagcaggagaatcgctcgaacccgggaggcagagattgcagtgagccaagatcgcgccaatgcactccagcatgggtgacagtgagactcggtctcaaaaaaaaaaaaaaaaaaaagaaagaaagaggctgggcgcggtggctcacacctgtaatcccagcactttgggaggctgaggagggcagatcacttgaggtcaggagttcgagatcagcctggtcaacgtggtgaaatcccatcggtactaaaaatataaaaattagccagatgtggtggcgcacgcttgtaattgcagctactcaggaggctgaggcaggagaatttcttgaacctagaaagtggccgagattgcgccatcctgggcgacagagcaagactctgtctcaaaaaaaaaaaaaaaaaaaaaaaaaaaaaaaaaaaaaaaaagaaaagaaaaaagaaaaagagacaaagaaagaaaaataaatgaggccagagctatgggaggattttaagtggaggagtgaccgtggggctgttgtgtgacactgctagctttacttgtgtgcctaagtggtcctttctcagaatcttactttcttttcgtataaaatggggcttagcattgccatctgcaggacaagtacaagtcctaataatagctgacatttctggaaggcttgccatgtgtactttttggcatgacatcattgggtcctcatcgcaaacctctgaggttgattctattttcatcctcctctgaaaaatgagtcactgcagtcagagagggaaggccacctgtccctgaccacacagctgatgtcaggcacgtgggtcctaagccttccagggcctgccccctgggtccccatcctccagcctggaggccccagggcacggcttccctgctcacacctcagtttccttgctatcctagaaacctaagggtagctgggcgcggcagctcatgcctgtaattccagcactttgggaggccgaggtggccaattaccagccagaggtcaggagttcgagaccagcctggccaacgtggtgaaaccccgtctctactaaagttacaaaaattagccgggcatggtggcaggtgcctgtaatcccggctactcaggaggctgaggcaggagaattgcttgaacctgagaggcagaggttgcagtgagccaagatcgcaccactgcgctccagcctggacgacagagtaactctgtcaataataataataataataataaataaactgctgggcacagtggctcaagcctctaatcccagtactttgggaggccgaggtgggcagattacctgaggttgggagtttgagaccagcctgaccaacatggagaaaccccgtctctactaaaaatagaaaattagccgggcgcggtggctcatgcctgtaatcccagctactcgggaggctgaggcaggagaattgcttgaacctgggaggcagaggttgcagtgagccgagatcacgccattgcattccagcctgggcaacaagagccaaactccgtctcaaaataaacaaacaaactaactaaagaagcctaacagtaaatggcagctggtgtgtatgtgaccctgttgctctgcttcctccagggacacggccaacacggtgaaggaaaagttcgggaggaagctttatgaatccttactggtgtgagtgccccaccctcagccctgtgccctcctaggccctgactaacccctctcattactccgttctcacatgcatgcccctgcttgccttgcagtgggagccttcccgacatgaacaagatgctggataaggaagacttcactatgatgaagagagccatctttgcaacgcaggtatggattggacctctgtgcagagagatagcagttcctggcagccccagaggcagcttgccaggccagctcagaccccagagattgctgggagtcctgctttctcctgcagccctttatgaagggatctattctgaggtccacagaagggcacgtgctgactcgtgcaacttcagttgctggaggcgaggcacttagggtgggctgttttctgtggcttattctgccacgagggttgctgggacgtgtggtttctctgggattctggggtcaatgagcaaaaggaagcttatttgtttatttatttttattatatttatttatttattttgagatggagtctcgctctgtcgcccaggctggagtgcagtggcacgatctcggctcactgcaacctctgcctcctgggttcaagcaatactcctgccttggcctcccaagtacctgggattacaggcacatgccaccacacccagctaatttatatatttttagtagaaacgagatttcaccatgttggccaggccagtctcaagctcctgacctcaagtgttctgtctgcctcagcctcccaaagtgctgagtttacaagcgtgagtccctgttcccagcccaaagggaagtttaaaaggaagtgtagaaatgtaataaagggccaggtgtggtggctcatgcctgtaatctcagcactttgagaggccaaagcaggaagatcacttgagaccaggaggctgaggctgcagtgagccatgactgtgccactgtacaccagcctgggggacagagccagatcccacactagccccccacccacccccaaaagaaagtcaaggcccagcttggtagctcacacctgtcagtattcccagcactttgggaggctgaggtgggaggatcgcttgagcccaggagtttgagaccagcctgggcaacatgatgaaaccctgtctctaccaaaaatacaaaaatgagctgggcatagtggcatacacctgttatcccagctacctgggaggctgaggccagaggatgccttgagctcaggaggtcaaggctgcagtgagctatgattatgccgctgcagtgagctatgattatgccactgcactccagactgggcaacagagcgaggcggacacatacacacacacacgaaagaaggaaaagaaagaaaagaaaaggtcaagacagccataaaagaattacaactctcagctctccttgggccttgctgcacacggctccacctctacccctaaaactgctgggagtggtagttttatcgggccactttagggacgcagtcaggaggtgagcaaaagcacccagcattctccagaaaacattgcaaactctcagcagctgcggtttcttatttccagaggctgtgtgttggtccccagacacccctgggaattgtagctgctttagatccctaacttcagagagaattggcatttataaaatgagaacttaccgtcattctattgagtctaggggcccaaaaaactacaactcccagcagcccctgctggctttctggggcattgacgcttcccttgtccagaggcttctgggagttagagttctttcattctctggggtccagggacttcagtacttgtggtctacattggggagccatttcctggggaatggaaacccagacagctaagttgtagaaacaaaacgtgaactgcttttggctaacctataatttccaatagtccctaaagcagcccacctcattatgaatgtttctgccatcgggatgctgggagttgtagtttctcgggcccctagactcagtagagtggtggtaagtaaaatctttaatgagctatgatggaacagttgctaagaactatacaccccagcagtcctcaggacttcctatcacactagctctgtcttgctccagagactcttggaaattgtggtttctttgggcctttgttagggttggaggtgaattttcttttttcttcttttttttttttttttttttttttgagacggagtctcgctctgtcgcccaggctggagtgcagtggcgcgatcttggctcacttcaacctccgcctcccggattcatgccgttctcctgccccagcctcctgaggcacccaccaccacgcctggctaattttttgtatttttagtagagacggggtttcaccgtgttagccaggatggtctcgatctcctgacctcgtcatcctcctgcctcggcctcccagagtgctgggattacaggcgtgagccaccacgcctggtggttggaagtgaattttcataaggaaactacaactcccagcagtctccaaagcttgctgccatattggcacagtctccacccccattgactggggatagttatatttcctttgggtttctttaataatagagcccagagtacagaacaaggtagctgcaactcctcatagtccttggaggtaagggagttgctaacacggctttgcctcaaagacagctgggaattctagttctttgaagctcatggtggttaagggaaggaaaacatttccaggccaggtgtggtggctcatgtctgtaatcccagcacttcgggaggccaaaacggatgcatcgcttgagcccatgagttcgagaccaacttggccaacatagtgaaaccccatctctattaaaaaaaaaaaaaaaaaaaattaggctgagcgcaatggctcacgcctgtaatcccagcactttgggaggccaaggcaggtggatcacgaggtcaggagatctagaccatcctgatttaacacagtgaaacctcgtctctactaaaaatacaaaaaattaaccgggcgtggtggtaggcgcctgtagttccagctactcgggaggctgaggcaggagaatggcatgatcctgggaggtggagcttgcagtgagccgagattgtgccactgcattccagcctgggcgacagagcgagactccgcctcaaaaataaataataataatttaaaagaaaagaaagaaaacctttacaggttgtactagtgggaatccacaactcctggtgatcacttgggtttgttgccccacgtccggtgcatttgtggtctccttggctcccccagggtccgcatggctcatgaggaagtagggagttctctgtcccttccatgaccccttacccttcttgtggctcctgcagcggcagtctttcccccctgtgtgcacccacaatatgctggatgactcctcagaccccatcctgaccaccatccgccgaatcggcctcttcaatagcagtgccgacagggtgaaggtgagggcatttggtcagggctgggttgggcacggcagagggaaggggttgggtcctgggcctcacacactccattcccctataagttttatcaaaggtattgctgtggttagaaagaaccctggcttctgaggcaggcagacctgggtttgaacattggtcctgccaaacacatgctgtgtgatgctagacaagctttttaccctctctgggccaccttgggatctcagggcttagctaaataaggcacttgggctgagtccttctttttcactaccaggtgattttccacccggagttcctctcctccacaagccccctgctccctgtggactatgaggagtttgtccgtggctgtcaccttggagtcttcccctcctactatgagccttggggctacacaccgggtgagtgtagtgggcaggggacagcgtggtcatggcatcctagtcagagctagcatgtcttggggcgaggcactggccttctctgagcctgttgccttctttgcaaaacaggaacaatggtaatcacctgttctgctacagcgcgtgcttttgtgatacaatagctcatgtattgagaaactagaagactgggttttgaggactagtgaactggatgtcacagttcacactgctttctcccagaaagcagtggccccccaaatagtggcaatgaaatgcacaattcacaaatacagccgagtgtggccaagtctcctcgtaagacgttcccagtgcccatcaactccaccttcctcttgttggttcacattagccagtgtttttaaagctaaataaggctgggcgtggtggctcatgcctgtaatcccagcacttggggaggccaagatgggcagatcatgaggtcaggagtttgagaccagcctggccaatatagtgaaaccctgtctctactaaaaatacaaaaattagccaggtgtggtggtacgcacctgtagtctcagctacttgggaggctgaggcagagaattgcttgaacttgggaggcggaggttgcagtgagctgagatcatgccactgcactccggcagtgctgtgctctgggtgacagagcgagactccgtctcaaaaaaaaaaaaaaaaaaaaattaaagctaaattagtgccatttcctaatttattagtaatttaacaagtcctgcaaactgtgcttgcatgataattaggagtcgcggtcagccccaattccctttttccataggccttgttttatagatagattgatagatagtactagtctgttttcacaccgctataaaaaaaaaatccaagactgggtaatttataacggaaagaggtgtaattgactcaccgttccacatgtctggagaggcctcagaaaactcagtcatggcagaaggggaagaaagaagcttcttctcatggcggcaggagggagaagtatgtgagggtgcaggaaaaacgaacatttataaaaccatgagatctcgtgagaattcactcactatcatgagaacagcggcgggggaaccacccccgtaatccaatcatttccctccctcgacacgtggggactacattttgagatgaaatttgggggggaacacagagtcaaactatatcataaatatatatttaatacattttactatttgtttcttttttgagatggggttattactctgttgccaaggctcacctggaactcctaggttcaggtgcttctgcctcagcctcttgagtatctgtcattataggtgcatcccaccatgcctgacagatgtattatatttagtgcctatttttgcagtttacagatgtttttaggaaaacttacataagaattatagatcaggccaggcatggtggctcacgcctataatccccacactttgggaggccgaggtgggaggatcacttgaggacatgagtttgagaccagcctggccaacatggtgaaaccctatctctactacaaatacaaaaattagctgggggtggtggcaggcacctgtaatcccagcccttcgggaagttgaggtgggagaatcgtttgaaccccagaggcagagattgcagtgagctgagatcgcgccactgcactccagcctgggcgacagagtcagaccctgtctaaaataataataataataattataggtcaggggcagtggctcacacctataatcctagcatcttggaaggctgaggtgggaggatagcttgagctcaggagttcaagaccagcctggacaacatggtgagaccctgtctctataaaagatttttttttttttttgagacagagtcttgctctattgccaggctggagtgcagtggcacagtcttggctcactgcaaccgcctcccgggttcaagtgattctcctgcctcagcctccagagtagctgggactataggcgcctgccaccacacccagctaatttttgtatttttattagacacagggtttcaccatgttggccaggatggtttttttttttttctcttcttgacctcgtgatccacccgccttggcctcccaaagtgctgggattacagacatgagccaccgtgcccggccttttttttttttatttttaattagccatgcgtagtgttgtgtgctcgtagtcccagcctcttgggtggctgaggtgggaggattgcttgagcctaggaggtcaaggctgcagtgagctatgatcaagccactgtcctccaggttgggtgataaagtgagaccttgtctcgagaaaaaaaaaaaaaaagcattgggccaggcgcagtggctcacacctataatttgaccactttgggaagccaagacggatggatcacctgaggtcaggagttcaagacctgcctggccaacatggtgaaaccctgtctatactaaaaaatacaataattaactgggcatggtggtgtgtgcctgtagtcccagctactcgggtggctaaggcacgaaaatcgcttgaacccgggaggtgaaggttgcagtgagccgagattgtgccactgcactacagcttgggtgacagagtgagaccctgtctcgaaagaaaaaaaaaaaagcataaaaatccatcaaaaaattataatacattccttgaactatttctcaaggttgttaaaggtattaaacaagacgtactagatgctctgtacacagggatactaataatgagagaccttatcttaccccccatcaaaatagactaagtccgtacaagatactggcacaagaaaggatcttacaggttgtgacaattaagtagaatgatggaccttggcccgatcctacaggttgtgagagttaagtcaaatggatattgggatgctcaataaattagcatccttatctcccaactatggactttcccgcatcttggagcaagagcaagtaattctcttttctgagcctcttagcgtcccctctgagaaatagggaaatgcacagcctggttgtgggggctcagcgacgctccgcacagcgcctggcacttggtacaggcgccagtagagggcggtgttgaggcgtctattgcgattaagacggtaggcgatgagggaagctaagtgggtctctctcctgccttcccgcagctgagtgcacggttatgggaatccccagtatctccaccaatctctccggcttcggctgcttcatggaggaacacatcgcagacccctcagcttacggtcagcgcccaggcccctgaggattctggaagggggcttgagaacagtgactcctggatcctgggaaggaggagcttgcacccttggagaccaggggcaccaggaaatgggggggctctcgggccgctgggagaggaggggtcctggtgccacagagtcctggagaccatgaagatctccccaggatgttttctgaacctgggtcctggcctccacaggtatctacattcttgaccggcggttccgcagcctggatgattcctgctcgcagctcacctccttcctctacagtttctgtcagcagagccggcggcagcgtatcatccagcggaaccgcacggagcgcctctccgaccttctggactggaaatacctaggccgggtaggacccccactttcctatcctctcctggcaaatcagcagctcctggctaggggtctttggtgcagggtgcagggtctcatcctacttgtaacagttgctgtccctttaagcaatgaaccaggccgatcgcgatggtggctcatggttgtaatcccagcactttgggaggcctaacaggaaggatcgcatgagctcaggagttaagagaccagcctgggcaacattttatttttactaaaaataaaaaaaaatcagccgagcgtgatggtgcgtgcctgtagtcccagctactggggaggctgaggtggtagggtcgcttgagccccggaggttgaggctgcagtgagccttgttcaaacccactgcactccatcctagagtaacagaacaagaccgtgtttaaaaagaaaaaaagaacattggtcaggcgttctcatttgcataagggtgtggccaaagaggagactcacttgagaattcggcccctgtttgaggggtatggctcagaaactgtccattcacaggtctgcttatctacataaggggtggagtcagatagatggattatttgtatggggtgcagcagtgtgctcatctgcataaagggtgtggccaagacccccatcatctgcctgggggtggggcctggctctcacatcctctgccccctctcctcctttcccctagtactatatgtctgcgcgccacatggcgctgtccaaggcctttccagagcacttcacctacgagcccaacgaggcggatgcggtgagtggaccctggatttctgcttagccaggagctaaagggctgggcttcggtggggtgggggtccctcctggagtgggaaggcctagaggtggctggatgcctgggtgggactgtgaggtcctcagctcaccctggtttgcacccacatcgccgtatgcaggcccaggggtaccgctacccacggccagcctcggtgccaccgtcgccctcgctgtcacgacactccagcccgcaccagagtgaggacgaggaggatccccggaacgggccgctggaggaagacggcgagcgctacgatgaggacgaggaggccgccaaggaccggcgcaacatccgtgcaccagagtggccgcgccgagcgtcctgcacctcctccaccagcggcagcaagcgcaactctgtggacacggccacctccagctcactcagcaccccgagcgagcccctcagccccaccagctccctgggcgaggagcgtaactaagtccgccccaccacactccccgcctgtcctgcctctctgctccagagagaggatgcagaggggtgctgctcctaaacccccgctccagatctgcactgggtgtggccccgcagtgcccccacccagtccgccaaacactccaccccctccagctccagtttccaagttcctgcactccagaatccacaaagccgtgcctttctctggctccagaatatgcataatcagcgccctggagtcccctgggcctggaccgcttcccagaggccaggaatctgccattactctgcggtggtgccagaggttttaggaaacctggcatggtgctttcaggtctggggcttttagagccccccgtgtggcttacaaattctacagcatacagagcaggccacgctcaggcccggcatgcgggccaccaagttctggaaaccacgtggtgtccctgcgaatggggcgatcaagtccagagccggggcactttcagagtttgaaggtaactgagagcagatggtcctccatttcaactccagaagtggggctctgggagggatgttctagccctccctggcatgtcagagccaggctctgcctggaggatccctccatccggctcctgtcatcccctacactttggccaagcaagaggtggtagaaccacttggctgctcattccttctggaggacacacagtctcagtccagatgccttcctgtctttctggccctttctggaccagatcctactcttcctttctaaatctgagatctccctccagggaatccgcctgcagaggacagagctggctgtcttcccccacccctaacctggcttattcccaactgctctgcccactgtgaaaccactaggttctaggtcctggcttctagatctggaaccttaccacgttactgcatactgatccctttcccatgatccagaactgaggtcactgggttctagaacccccacatttacctcgaggctcttccatccccaaactgtgccctgccttcagctttggtgaaagggagggcccctcatgtgtgctgtgctgtgtctgcaccgcttggtttgcagttgagaggggagggcaggaggggtgtgattggagtgtgtccggagatgagatgaaaaaaatacatctatatttaagaatcccaggtgtggtcagacatgacacttggtgggcctggctgtgacgtccccccagtaccaccccttccctggtctgagctcatcctcttattctagagagacttggggggtctccctatgttgcttaggctggtctcaacctcctgggctcaagtgatcgtcctgccttggcctcccaaagtgctgggattacaggcttgaaccaccctgccccagacccaggggaaggagaaggggaagagtcagagctgacacagcagttttgcagcttggacacagcaagtgctttttaaggcctgttttaagcttcttcagttcccatttggagaatccggcccaaggtttcacatcccatttctgtgatgaaccctaaaccccttgtccagcattgcttgggcatcacgtgttcctggctgctcagctccactggccagttcctctcagtgtgcgcctcacattgggatcactcaagctcgggccataatctcttaaactctggagcaaagacctcagtgagccccccggttgccatgcaagttgcatagcaatagcgtcaatgtgcccatataaagggcctaataattccaactgcctcccagaattgaggcttttttcttttttgagacagagtcttactctgttgcccaagctggagcacagtggcgccatctcagctcaccgcaacttccgtgaggcagacatgttttttgtttgagatgggagttccactctttttgcccaggctggagtgcaatgacgccatctcagctcactgcaacctccgcctcccagattcaagcgattctcctacctcaacctcctgactagctgggattacaggtatgagccaccacacccagctaattttgtattttagtagagacggggtttcttcatgttggtcaggctggtctcgaactcctgacatcaggtgagcgacccacctggccccataatcactcgttaaatagggctgtttctggctgagacctaacatctgtttctagataccaacctaggtcttaattcaactccctggagtctatctctaccatctgggagggacagttaacagccaggcaagcagagaaccctcaggttcaacactacccagatagcctcttgtcttaggacagttaagaaaatgggagatggggccaactcctctttcactggcatcccaacccagccagaagctttatgagaacagatcccacctcatcttccacaccacacacaaaaccaccagctgcatcaaaaagctttatttccatttggtccaaggcttgttaggatagttaagaaagctgcctattggctggagggagaggcttaggcagaagccctattactttgcaaggggcccttcagaagtcgctgggctcagaaggctcttagtcgtgcttgagagtgagcctttcgaagagatactcgcccagcccagcctccgggccacccagcctgtggaggttggtcaggtggtcacccatcttcttgataagcttcacttcctcatctaggaagtgagtctccaggaagtcacagagctgagagagaagagggagaaattagaaacccaatccctggaaaagagggggagagagcagccagttgcagattaaaatgtgacaggtgtgaaatgaggctctgaaggaaatttgcccaaagggagcagaggcttgaggggtatggttgggtagccatggatgcagcgggtacgtacatgggggtccgtgcgggcagaacccagggcatgaagatccaaaagggcctggttcagctttttctccagggccatggcagctttcatggcgtctggggttttaccccactcatcttcagctggcttctatatagaagggagaaatggctcagaatacacacactcggcacatagaactaaacctacatttcccaagagtcgttgggggtcagaggcccaggccacaaagacatgtgacagcttgtatttatcactctccgcaccctgtcctagctcttacagctatacgtccctaagaccacgcagcggtgtggactgccgcgtgttgtgggcactgcacgaggtgcgcctctatttccagcggttaagagggctcacaagaccgaactcaatctcccagaagcccacgcgacacctacgccctcaaatcaaggctcctcgcgcgcacggcctgctgggagatgtagtccattacccacacactagttaccttgatgtcctggaagagagcgcggccgccacgctggttttgcatcttcaggagacgctcgtagccctcgcgcttctcctcggccaattcgcggaagaagtggctcacgccttccagagccacatcatcgcggtcgaaatagaagccctacgggaagagatggaggcaacaatggttagcgggaggcgaggccaaggggactccgccctctgtttacccgaccgcacaaagaaggctggcgcgtgcagtgagggccggaggtgcgcagctggaggaaattagggccaggggcgtcctggggactcaccagagagaggtaggtgtaggaggcctgcaggtacaaattgaccaggctgttgacggctgcctccacgtcggtggaataattctgacgaatctgggagctcatggttggttggcaagaaggagctaaccacaaaaacggtgctggcaggtcccagaagcaggagatggccgagaagatggtcccggaggttgcaagcggagaggaaatcggagggcggtcggaggctggaagagagtccccggatctgttccgtccaaacactgttgaagcaagagacagacccgcgggaccgccgaactgcgaggggacgtggctagggcggcttcttttatggtgcgccggccctcggaggcagggcgctcggggagggctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctggggctgattctggaaatgcaggggttcgtgggcgggagggtgggggctggggtccctctgggaggctagagttttatg

SEQ ID NO: 7 - DNA, 22 bp, synthetic construct
  misc_feature 1..22: note = Primer
  source 1..22: mol_type = other DNA
tgatgaagagagccatctttgc

SEQ ID NO: 8 - DNA, 21 bp, synthetic construct
  misc_feature 1..21: note = Primer
  source 1..21: mol_type = other DNA
aggagtcgtccagcatgttgt

SEQ ID NO: 9 - DNA, 24 bp, synthetic construct
  misc_feature 1..24: note = Probe
  source 1..24: mol_type = other DNA
actcagcggcagtctttcccacca

SEQ ID NO: 10 - DNA, 20 bp, synthetic construct
  misc_feature 1..20: note = Synthetic oligonucleotide
  source 1..20: mol_type = other DNA
ccgactcaggtagggtgagc

SEQ ID NO: 11 - DNA, 20 bp, synthetic construct
  misc_feature 1..20: note = Synthetic oligonucleotide
  source 1..20: mol_type = other DNA
cttggtgaccggtagagtta

SEQ ID NO: 12 - DNA, 20 bp, synthetic construct
  misc_feature 1..20: note = Synthetic oligonucleotide
  source 1..20: mol_type = other DNA
gagaggcatggctactgcgg

SEQ ID NO: 13 - DNA, 20 bp, synthetic construct
  misc_feature 1..20: note = Synthetic oligonucleotide
  source 1..20: mol_type = other DNA
cggctgagaggcatggctac

SEQ ID NO: 14 - DNA, 20 bp, synthetic construct
  misc_feature 1..20: note = Synthetic oligonucleotide
  source 1..20: mol_type = other DNA
tcttccaatcctggaagcga

SEQ ID NO: 15 - DNA, 20 bp, synthetic construct
  misc_feature 1..20: note = Synthetic oligonucleotide
  source 1..20: mol_type = other DNA
atggtcccaccagatagtag

SEQ ID NO: 16 - DNA, 20 bp, synthetic construct
  misc_feature 1..20: note = Synthetic oligonucleotide
  source 1..20: mol_type = other DNA
cgtgtatggtcccaccagat

SEQ ID NO: 17 - DNA, 20 bp, synthetic construct
  misc_feature 1..20: note = Synthetic oligonucleotide
  source 1..20: mol_type = other DNA
aggtgttgagcctcgattgc

SEQ ID NO: 18 - DNA, 20 bp, synthetic construct
  misc_feature 1..20: note = Synthetic oligonucleotide
  source 1..20: mol_type = other DNA
tgactgtattggctgtgtcc

SEQ ID NO: 19 - DNA, 20 bp, synthetic construct
  misc_feature 1..20: note = Synthetic oligonucleotide
  source 1..20: mol_type = other DNA
ctccttgactgtattggctg

SEQ ID NO: 20 - DNA, 20 bp, synthetic construct
  misc_feature 1..20: note = Synthetic oligonucleotide
  source 1..20: mol_type = other DNA
gtagagcttcctcccaaatt

SEQ ID NO: 21 - DNA, 20 bp, synthetic construct
  misc_feature 1..20: note = Synthetic oligonucleotide
  source 1..20: mol_type = other DNA
cctccgatccagaatgtaaa

SEQ ID NO: 22 - DNA, 20 bp, synthetic construct
  misc_feature 1..20: note = Synthetic oligonucleotide
  source 1..20: mol_type = other DNA
acttccaatctagcaagtcc

SEQ ID NO: 23 - DNA, 20 bp, synthetic construct
  misc_feature 1..20: note = Synthetic oligonucleotide
  source 1..20: mol_type = other DNA
tcccgtggctcttcctcatc

SEQ ID NO: 24 - DNA, 20 bp, synthetic construct
  misc_feature 1..20: note = Synthetic oligonucleotide
  source 1..20: mol_type = other DNA
tgtggaggaggaacaggagg

SEQ ID NO: 25 - DNA, 20 bp, synthetic construct
  misc_feature 1..20: note = Synthetic oligonucleotide
  source 1..20: mol_type = other DNA
tgccacctgtggaggaggaa

SEQ ID NO: 26 - DNA, 20 bp, synthetic construct
  misc_feature 1..20: note = Synthetic oligonucleotide
  source 1..20: mol_type = other DNA
ctggagggcccagtgtccac

SEQ ID NO: 27 - DNA, 20 bp, synthetic construct
  misc_feature 1..20: note = Synthetic oligonucleotide
  source 1..20: mol_type = other DNA
gtgagctggagggcccagtg

SEQ ID NO: 28 - DNA, 20 bp, synthetic construct
  misc_feature 1..20: note = Synthetic oligonucleotide
  source 1..20: mol_type = other DNA
cataggccctctgcgagagg

SEQ ID NO: 29 - DNA, 20 bp, synthetic construct
  misc_feature 1..20: note = Synthetic oligonucleotide
  source 1..20: mol_type = other DNA
atctgcataggccctctgcg

SEQ ID NO: 30 - DNA, 20 bp, synthetic construct
  misc_feature 1..20: note = Synthetic oligonucleotide
  source 1..20: mol_type = other DNA
ttcaggcaccctcccatctg

SEQ ID NO: 31 - DNA, 20 bp, synthetic construct
  misc_feature 1..20: note = Synthetic oligonucleotide
  source 1..20: mol_type = other DNA
actcaagagtctggagtggg

SEQ ID NO: 32 - DNA, 20 bp, synthetic construct
  misc_feature 1..20: note = Synthetic oligonucleotide
  source 1..20: mol_type = other DNA
ggctggagtgtctgaaacag

SEQ ID NO: 33 - DNA, 20 bp, synthetic construct
  misc_feature 1..20: note = Synthetic oligonucleotide
  source 1..20: mol_type = other DNA
tggagctcaagggctggagt

SEQ ID NO: 34 - DNA, 20 bp, synthetic construct
  misc_feature 1..20: note = Synthetic oligonucleotide
  source 1..20: mol_type = other DNA
ccaagaaaggcacggcggcg

SEQ ID NO: 35 - DNA, 20 bp, synthetic construct
  misc_feature 1..20: note = Synthetic oligonucleotide
  source 1..20: mol_type = other DNA
ctggagactccagatcagtg

SEQ ID NO: 36 - DNA, 20 bp, synthetic construct
  misc_feature 1..20: note = Synthetic oligonucleotide
  source 1..20: mol_type = other DNA
aaacaatggcagatgcctgg

SEQ ID NO: 37 - DNA, 20 bp, synthetic construct
  misc_feature 1..20: note = Synthetic oligonucleotide
  source 1..20: mol_type = other DNA
cctaaaacctctggcattga

SEQ ID NO: 38 - DNA, 20 bp, synthetic construct
  misc_feature 1..20: note = Synthetic oligonucleotide
  source 1..20: mol_type = other DNA
cctggaagccaataaaccag

SEQ ID NO: 39 - DNA, 20 bp, synthetic construct
  misc_feature 1..20: note = Synthetic oligonucleotide
  source 1..20: mol_type = other DNA
gccacagcctggaagccaat

SEQ ID NO: 40 - DNA, 20 bp, synthetic construct
  misc_feature 1..20: note = Synthetic oligonucleotide
  source 1..20: mol_type = other DNA
gccacacagaatccaacatg

SEQ ID NO: 41 - DNA, 20 bp, synthetic construct
  misc_feature 1..20: note = Synthetic oligonucleotide
  source 1..20: mol_type = other DNA
tcgggaagccacacagaatc

SEQ ID NO: 42 - DNA, 20 bp, synthetic construct
  misc_feature 1..20: note = Synthetic oligonucleotide
  source 1..20: mol_type = other DNA
cctgaaatgtcctaactctg

SEQ ID NO: 43 - DNA, 20 bp, synthetic construct
  misc_feature 1..20: note = Synthetic oligonucleotide
  source 1..20: mol_type = other DNA
ttaatccctgaaatgtccta

SEQ ID NO: 44 - DNA, 20 bp, synthetic construct
  misc_feature 1..20: note = Synthetic oligonucleotide
  source 1..20: mol_type = other DNA
aatctgtcgacagagctact

SEQ ID NO: 45 - DNA, 20 bp, synthetic construct
  misc_feature 1..20: note = Synthetic oligonucleotide
  source 1..20: mol_type = other DNA
gacaagcaatctgtcgacag

SEQ ID NO: 46 - DNA, 20 bp, synthetic construct
  misc_feature 1..20: note = Synthetic oligonucleotide
  source 1..20: mol_type = other DNA
tgtgtatcaccgcaccaggt

SEQ ID NO: 47 - DNA, 20 bp, synthetic construct
  misc_feature 1..20: note = Synthetic oligonucleotide
  source 1..20: mol_type = other DNA
gaaatggaggaccgtgagca

SEQ ID NO: 48 - DNA, 20 bp, synthetic construct
  misc_feature 1..20: note = Synthetic oligonucleotide
  source 1..20: mol_type = other DNA
tgctcctttgaagaacacac

SEQ ID NO: 49 - DNA, 20 bp, synthetic construct
  misc_feature 1..20: note = Synthetic oligonucleotide
  source 1..20: mol_type = other DNA
gcagaaaggtgtctggtcca

SEQ ID NO: 50 - DNA, 20 bp, synthetic construct
  misc_feature 1..20: note = Synthetic oligonucleotide
  source 1..20: mol_type = other DNA
aaggcagaaaggtgtctggt

SEQ ID NO: 51 - DNA, 20 bp, synthetic construct
  misc_feature 1..20: note = Synthetic oligonucleotide
  source 1..20: mol_type = other DNA
tgacagacattctgccctca

SEQ ID NO: 52 - DNA, 20 bp, synthetic construct
  misc_feature 1..20: note = Synthetic oligonucleotide
  source 1..20: mol_type = other DNA
agtgggctgagcacttgtgg

SEQ ID NO: 53 - DNA, 20 bp, synthetic construct
  misc_feature 1..20: note = Synthetic oligonucleotide
  source 1..20: mol_type = other DNA
agccactgggacccagaacc

SEQ ID NO: 54 - DNA, 20 bp, synthetic construct
  misc_feature 1..20: note = Synthetic oligonucleotide
  source 1..20: mol_type = other DNA
ttcaagaagccggtgggctc

SEQ ID NO: 55 - DNA, 20 bp, synthetic construct
  misc_feature 1..20: note = Synthetic oligonucleotide
  source 1..20: mol_type = other DNA
gcagaaaggcctcgaggtac

SEQ ID NO: 56 - DNA, 20 bp, synthetic construct
  misc_feature 1..20: note = Synthetic oligonucleotide
  source 1..20: mol_type = other DNA
ccccccagggcctaggacgc

SEQ ID NO: 57 - DNA, 20 bp, synthetic construct
  misc_feature 1..20: note = Synthetic oligonucleotide
  source 1..20: mol_type = other DNA
acagcattgagtctgccatc

SEQ ID NO: 58 - DNA, 20 bp, synthetic construct
  misc_feature 1..20: note = Synthetic oligonucleotide
  source 1..20: mol_type = other DNA
tggcctgactggatgctgga

SEQ ID NO: 59 - DNA, 20 bp, synthetic construct
  misc_feature 1..20: note = Synthetic oligonucleotide
  source 1..20: mol_type = other DNA
attgatctaactctgtccca

SEQ ID NO: 60 - DNA, 20 bp, synthetic construct
  misc_feature 1..20: note = Synthetic oligonucleotide
  source 1..20: mol_type = other DNA
atccttggattaaaagagtg

SEQ ID NO: 61 - DNA, 20 bp, synthetic construct
  misc_feature 1..20: note = Synthetic oligonucleotide
  source 1..20: mol_type = other DNA
gaccaaaactcccagatttc

SEQ ID NO: 62 - DNA, 20 bp, synthetic construct
  misc_feature 1..20: note = Synthetic oligonucleotide
  source 1..20: mol_type = other DNA
agccacatgtagggaccaca

SEQ ID NO: 63 - DNA, 20 bp, synthetic construct
  misc_feature 1..20: note = Synthetic oligonucleotide
  source 1..20: mol_type = other DNA
catgcttcatttctttattg

SEQ ID NO: 64 - DNA, 20 bp, synthetic construct
  misc_feature 1..20: note = Synthetic oligonucleotide
  source 1..20: mol_type = other DNA
ggcccatgcttcatttcttt

SEQ ID NO: 65 - DNA, 20 bp, synthetic construct
  misc_feature 1..20: note = Synthetic oligonucleotide
  source 1..20: mol_type = other DNA
tcagagatagccagagagag

SEQ ID NO: 66 - DNA, 20 bp, synthetic construct
  misc_feature 1..20: note = Synthetic oligonucleotide
  source 1..20: mol_type = other DNA
ccctactgtctcatgactta

SEQ ID NO: 67 - DNA, 20 bp, synthetic construct
  misc_feature 1..20: note = Synthetic oligonucleotide
  source 1..20: mol_type = other DNA
gaggcctcagcaaatgccag

SEQ ID NO: 68 - DNA, 20 bp, synthetic construct
  misc_feature 1..20: note = Synthetic oligonucleotide
  source 1..20: mol_type = other DNA
cctccagcaatgtattttaa

SEQ ID NO: 69 - DNA, 20 bp, synthetic construct
  misc_feature 1..20: note = Synthetic oligonucleotide
  source 1..20: mol_type = other DNA
aggaatcagagggttctgtg

SEQ ID NO: 70 - DNA, 20 bp, synthetic construct
  misc_feature 1..20: note = Synthetic oligonucleotide
  source 1..20: mol_type = other DNA
agccctctcttttatgacaa

SEQ ID NO: 71 - DNA, 20 bp, synthetic construct
  misc_feature 1..20: note = Synthetic oligonucleotide
  source 1..20: mol_type = other DNA
acaagctaaagacttaaact

SEQ ID NO: 72 - DNA, 20 bp, synthetic construct
  misc_feature 1..20: note = Synthetic oligonucleotide
  source 1..20: mol_type = other DNA
gatttgcaagtgactctcaa

SEQ ID NO: 73 - DNA, 20 bp, synthetic construct
  misc_feature 1..20: note = Synthetic oligonucleotide
  source 1..20: mol_type = other DNA
tggattccctctgtagatca

SEQ ID NO: 74 - DNA, 20 bp, synthetic construct
  misc_feature 1..20: note = Synthetic oligonucleotide
  source 1..20: mol_type = other DNA
tgtctctagctctgacaaca

SEQ ID NO: 75 - DNA, 20 bp, synthetic construct
  misc_feature 1..20: note = Synthetic oligonucleotide
  source 1..20: mol_type = other DNA
ccagatgctatttctagatt

SEQ ID NO: 76 - DNA, 20 bp, synthetic construct
  misc_feature 1..20: note = Synthetic oligonucleotide
  source 1..20: mol_type = other DNA
actgctggagtccccagcaa

SEQ ID NO: 77 - DNA, 20 bp, synthetic construct
  misc_feature 1..20: note = Synthetic oligonucleotide
  source 1..20: mol_type = other DNA
cctataggactatccaggaa